(19) United States
(12) Patent Application Publication
(10) Pub. No.: US 2022/0008452 A1
(43) Pub. Date: Jan. 13, 2022

(54) COMPOSITIONS AND METHODS FOR TREATING OR PREVENTING NASH, NAFLD, DIABETES, ATHEROSCLEROSIS, AND/OR OBESITY

(71) Applicant: YALE UNIVERSITY, NEW HAVEN, CT (US)

(72) Inventors: Hyung CHUN, Guilford, CT (US); Takaomi ADACHI, East Haven, CT (US)

(21) Appl. No.: 17/311,502

(22) PCT Filed: Dec. 6, 2019
(86) PCT No.: PCT/US19/64974

§ 371 Date: Jun. 7, 2021

Related U.S. Application Data
(60) Provisional application No. 62/776,899, filed on Dec. 7, 2018.

(51) Int. Cl. A61K 31/7105; A61P 3/04; A61P 3/10; A61P 9/10

(52) U.S. Cl. CPC A61K 31/7105; A61P 9/10; A61P 3/10; A61P 3/04

(57) ABSTRACT

In various aspects and embodiments the invention provides compositions and methods useful in the treatment of certain metabolic diseases, such as but not limited to NASH, NAFLD, diabetes, atherosclerosis, and/or obesity.

### CROSS-REFERENCE TO RELATED APPLICATIONS

The present application claims priority under 35 U.S.C. § 119(e) to U.S. Provisional Patent Application No. 62/776,899, filed Dec. 7, 2018, which is incorporated herein by reference in its entirety.

### STATEMENT REGARDING FEDERALLY SPONSORED RESEARCH OR DEVELOPMENT

This invention was made with government support under HL 113005 awarded by the National Institutes of Health. The government has certain rights in the invention.

### BACKGROUND OF THE INVENTION

Obesity and complications such as non-alcoholic steatoheapatitis (NASH), non-alcoholic fatty liver disease (NAFLD), diabetes and artherosclerosis are a significant cause of mortality and morbidity.

Steatohepatitis (also known as fatty liver disease) is a type of liver disease, characterized by liver inflammation with concurrent fat accumulation in the liver.

Steatohepatitis is characterized microscopically by hepatic fat accumulation (steatosis), mixed lobular inflammation, ballooning degeneration of hepatocytes (sometimes with identifiable Mallory bodies), glycogenated hepatocyte nuclei, and pericellular fibrosis. The “chicken wire” pattern of the pericellular fibrosis, which affects portal areas only secondarily in later stages, is very characteristic and is identified on trichrome stains.

Classically seen in alcoholics as a part of alcoholic liver disease, steatohepatitis is also frequently found in people with diabetes and obesity and is related to metabolic syndrome. When not associated with excessive alcohol intake, it is referred to as NAFLD. NAFLD is a key factor in the pathogenesis of type 2 diabetes (T2D) and affects one in three Americans. NAFLD is also a key predisposing factor for the development of NASH, cirrhosis, and hepatocellular carcinoma. Further, NAFLD-induced NASH may soon surpass hepatitis C and alcoholic cirrhosis as the most common indication for liver transplantation in the USA. NASH is also associated with lysosomal acid lipase deficiency.

NASH is commonly associated with metabolic syndrome (obesity, dyslipidemia and insulin resistance). Further progression of the disease is probably caused by chronic inflammation and reactive oxygen species formation. Metabolically induced liver inflammation recruits additional inflammatory components (neutrophils, AP-1 pathway) and causes NASH. A retrospective cohort study concluded that liver failure is the main cause of morbidity and mortality in NASH-associated cirrhosis. No treatment has yet emerged as the “gold standard” for NASH.

There is a need in the art for new therapies to treat and prevent these conditions. The present disclosure addresses this need.

### SUMMARY OF THE INVENTION

In one aspect, the invention provides a method of treating non-alcoholic steatohepatitis (NASH) in a subject, wherein the method comprises administering to the subject a therapeutically effective amount of at least one miRNA335 inhibitor.

In another aspect, the invention provides a method of treating non-alcoholic fatty liver disease (NAFLD) in a subject, wherein the method comprises administering to the subject a therapeutically effective amount of at least one miRNA335 inhibitor.

In yet another aspect, the invention provides a method of treating diabetes in a subject, wherein the method comprises administering to the subject a therapeutically effective amount of at least one miRNA335 inhibitor.

In yet another aspect, the invention provides a method of treating atherosclerosis in a subject, wherein the method comprises administering to the subject a therapeutically effective amount of at least one miRNA335 inhibitor.

In yet another aspect, the invention provides a method of treating obesity in a subject, wherein the method comprises administering to the subject a therapeutically effective amount of at least one miRNA335 inhibitor.

In certain embodiments, the at least one miRNA335 inhibitor comprises an antagomir.

In certain embodiments, the subject is a mammal

In certain embodiments, the subject is a human.

In certain embodiments, the at least one miRNA335 inhibitor is formulated as a pharmaceutical composition further comprising at least one pharmaceutically acceptable carrier.

### BRIEF DESCRIPTION OF THE DRAWINGS

The following detailed description of exemplified embodiments of the invention will be better understood when read in conjunction with the appended drawings. For the purpose of illustrating the invention, certain embodiments are shown in the drawings. It should be understood, however, that the invention is not limited to the precise arrangements and instrumentalities of the embodiments shown in the drawings.

FIGS. 1A-1B show that miR335−/− mice gain less weight on a high fat diet. FIG. 1A is a timeline of the diet of control and knockout mice. FIG. 1B is a graph of mouse body weight.

FIGS. 2 and 3 show images depicting fat accumulation in the liver of control and miR335−/− knockout animals on high fat diet. miR335−/− mice have decreased fat accumulation in the liver after high fat diet.

FIG. 4 depicts a graph showing that miR335−/− mice have decreased fat accumulation in the liver on a high fat diet.

FIGS. 5 and 6 are images showing that liver histology is preserved in miR335−/− mice.

FIG. 7 is a graph showing that serum alanine amino transferase (ALT) elevation due to high fat diet is attenuated in miR335−/− mice.

FIG. 8 shows that plasma lipid profile is not changed in miR335−/− mice.

FIG. 9 shows that fatty acid uptake into the liver is decreased in miR335−/− mice.

FIG. 10 shows that fatty acid uptake into circulation is not different between wild type and miR335−/− mice. The mice are fed with normal chow in this experiment.

FIG. 11 shows that genes related to fatty acid transfer are downregulated in the livers of miR335−/− mice. These genes are downstream genes of LXR. The mice are subject to 6 weeks of 60% HFD.

FIG. 12 depicts a Western blot corresponding to the graph in FIG. 11. Genes related to fatty acid transfer are downregulated in the livers of miR335−/− mice.

FIG. 13 is a network showing that hepatic steatosis is predicted to be inhibited in the livers of miR335−/− mice.

FIG. 14 is a schematic outlining time course of an experiment comparing animals administered miR335 inhibitors to control, on a high fat diet. The mice were injected 5 mg/kg of miR335 inhibitor or its control twice a week for 4 weeks. Nonproprietary in vivo grade miRNA inhibitor purchased from Qiagen/Exiqon. Sequence: ACATTTTTCGTTATTGCTCTTGA (SEQ ID NO:3; 100% complementary to mi355-5p).

FIG. 15 shows the specifics of the high fat die for the animals (60% high fat diet).

FIG. 16 shows that the weight gain associated with a high fat diet (60% high fat diet) is suppressed in animals taking miR335 inhibitors. The mice are subject to 6 weeks of 60% HFD. 5 mg/kg of miR335 inhibitor and its control was given to mice by IP injection. 2 weeks after, HFD was started.

FIG. 17 shows that liver weight is decreased in animals taking miR335 inhibitors.

FIGS. 18-20 show that fat accumulation in the liver is attenuated in animals taking miR335 inhibitors.

FIG. 21 and FIG. 22 show that liver histology is preserved in animals taking miR335 inhibitors.

FIG. 23 shows that the elevation in serum ALT associated with high fat diet is attenuated in animals taking miR335 inhibitors.

FIG. 24 shows that MiR-335 knockout mice have better glucose utilization and insulin sensitivity than wild-type mice.

FIGS. 25A-25I show that MiR-335 is downregulated by statin stimulation of endothelial cells and induces activation of NF-kB. (FIG. 25A) Volcano plot demonstrating microRNA expression profiling analysis in HUVECs treated with simvastatin. MiR-335-5p and miR-335-3p are marked with arrowheads. (FIG. 25B) Relative expression levels of miR-335-5p in response to atorvastatin in HCMECs and HUVECs. *P<0.05, ***P<0.001 vs. control. (FIG. 25C) Relative mRNA expression levels of MEST in response to atorvastatin in HCMECs and HUVECs. **P<0.01, ***P<0.001 vs. control. (FIG. 25D) Relative expression levels of miR-335-5p and MEST in response to KLF2 or KLF4 knockdown (siKLF2 or siKLF4) in HUVECs. **P<0.01, ***P<0.001 vs. control. (FIG. 25E) Flow cytometry analyses of ICAM-1 and E-SELECTIN expression in HUVECs in response to miR-335 overexpression. control, miR-335 mimic. **P<0.01, ***P<0.001 vs. control. (FIG. 25F) Monocyte adhesion assay using fluorescently labeled THP-1 monocytes on HCMECs and HUVECs transfected with either miR-335 or control mimic. White dots are fluorescent labeled monocytes. ***P<0.001 vs. control. Scale bar: 100 μm. (FIG. 25G) Schematic of the highest scoring IPA network of gene expression profiling of HUVECs subjected to mir-335 overexpression. Upregulated genes identified in the expression profile with at least 1.5 fold increased expression appear red and downregulated genes with at least 1.5 fold decreased expression appear green. Genes shaded gray represent genes from the profile that did not exceed the 1.5 fold expression change cutoff. Gene symbols with no fill correspond to genes from the IPA Knowledge Base and are not part of the mir-335 overexpression profile. Unbroken lines represent direct interactions, and dashed lines indirect interactions. (FIG. 25H) Luciferase assay using HUVECs transfected with an NF-kB promoter driven luciferase reporter co-transfected with either miR-335 or control mimic. *P<0.05 vs. control. (FIG. 25I) Flow cytometry analyses of HUVECs transduced with an NF-κB-GFP lentiviral reporter transfected with either miR-335 or control mimic. **P<0.01 vs. control.

FIGS. 26A-26G show that CHFR is a direct target of miR-335 and negatively regulates NF-κB activity. (FIG. 26A) Relative mRNA and protein expression levels of CHFR in response to miR-335 overexpression in HCMECs and HUVECs. **P<0.01 vs. control. (FIG. 26B) Relative mRNA and protein expression levels of CHFR in response to atorvastatin stimulation of HCMECs and HUVECs. **P<0.01 vs. control. (FIG. 26C) Targeting of the CHFR 3′ UTR by overexpression of miR-335 in HUVECs. Luciferase activity data for constructs with the wildtype (WT) and mutant 3′ UTR constructs are shown. *P<0.01 vs. control. (FIG. 26D) Representative images and quantification of monocyte adhesion assay in HCMECs and HUVECs with either control or CHFR knockdown (siCHFR). The white cells are fluorescent labeled monocytes on a layer of unlabeled ECs. ***P<0.001 vs. control. Scale bar: 100 μm. (FIG. 26E) Flow cytometry analyses of ICAM-1 and E-SELECTIN expression in HUVECs in response to CHFR knockdown. **P<0.01, ***P<0.001 vs. control. (FIG. 26F) Flow cytometry analyses of HUVECs transduced with an NF-κB-GFP lentiviral reporter transfected with either CHFR targeting siRNA or control. **P<0.01 vs. control. (FIG. 26G) Flow cytometry to determine levels of ICAM-1 and E-SELECTIN expression in HUVECs in response to miR-335 and CHFR overexpression. **P<0.01 vs. control vector.

FIGS. 27A-27E show that miR-335 promotes atherosclerosis and endothelial inflammation. (FIG. 27A) Representative images of in situ hybridization in mouse aorta, skeletal muscle (Sk. Musc.) and white adipose tissue (WAT) stained for miR-335 and miR-126. MicroRNAs are stained as purple color in the nuclei. Lu: lumen. Scale bar: 20 μm. (FIG. 27B) Representative images and quantification of Oil Red O stained aortas from Apoe−/− and Apoe:Mir335 DKO mice on high-fat diet for 16 weeks (n=5 per group). ***P<0.001 vs. Apoe−/−. (FIG. 27C) Representative images and quantifications of H and E stained aortic sinus plaque area and valve thickness (n=5 per group). Scale bar: 200 μm. *P<0.05. **P<0.01 vs. Apoe−/−. (FIG. 27D) Representative images of immunohistochemistry for ICAM-1 in mouse aorta. Scale bar: 10 μm. (FIG. 27E) Representative images of immunohistochemistry for E-SELECTIN in mouse aorta. Scale bar: 10 μm

FIGS. 28A-28N show that miR-335 deletion promotes metabolic homeostasis by inhibiting trans-endothelial fatty acid transfer. (FIG. 28A) Body weights of Mir335−/− mice compared to control littermates. n=3-4 mice per normal chow group. n=12-13 mice after 6 weeks of 60% high-fat diet (HFD) group. **P<0.01 vs. control. (FIG. 28B) Intraperitoneal glucose tolerance test (IPGTT) of Mir335−/− mice and control littermates fed normal chow or 6 weeks of 60% HFD. (Control: n=13, Mir335−/−: n=12 for normal chow, n=12 per group for 6 weeks of 60% HFD). *P<0.05, **P<0.01 vs. control. (FIG. 28C) Intraperitoneal insulin tolerance testing of Mir335−/− mice and their control littermates after 6 weeks of 600% HFD. (Control: n=8, Mir335−/−: n=7). *P<0.05, **P<0.01 vs. control. (FIG. 28D) Plasma insulin levels and HOMA-IR in Mir335−/− mice and control littermates after 6 weeks of 60% HFD. (Control: n=8, Mir335−/−: n=7). **P<0.01 vs. control. (FIG. 28E) Relative protein expression levels of FABP4 in response to miR-335 overexpression in HUVECs. *P<0.05 vs. control. (FIG. 28F) Representative images of immunohistochemistry in mouse skeletal muscle stained for FABP4, CHFR and CD31. Scale bar: 20 μm. (FIG. 28G) Relative protein expression levels of FABP4 and CHFR in skeletal muscle of Mir335−/− mice and control littermates after 6 weeks of 60% HFD (n=8 per group). *P<0.05, **P<0.01 vs. control. (FIG. 28H) Relative mRNA and protein expression levels of FABP4 in response to CHFR silencing (siCHFR) and parthenolide (NF-κB inhibitor) stimulation in HUVECs. **P<0.01 vs. control. (FIG. 28I) Assessment of the FA intake in HCMECs in response to miR-335 overexpression or CHFR silencing. **P<0.01 vs. control. (FIG. 28J) Assessment of the FA transfer across a confluent HCMEC layer, as measured by determination of BODIPY in the bottom well of a Transwell plate, in response to miR-335 overexpression or CHFR silencing. *P<0.05, **P<0.01, ***P<0.001 vs. control. (FIG. 28K) Representative images and quantification of Oil Red O staining of skeletal muscle in Mir335−/− mice and control littermates (n=5 per group). Scale bar: 60 μm. (FIG. 28L) Skeletal muscle fatty acid and triglyceride content of Mir335−/− and control littermates (n=3-4 per group). *P<0.05 vs. control. (FIG. 28M) Tissue uptake of fluorescently labeled FA in the skeletal muscle (Sk. Musc.), white adipose tissue (WAT), brown adipose tissue (BAT), liver, and blood of Mir335−/− mice and control littermates at 6 h after oral gavage (n=5 for each timepoint). *P<0.05, **P<0.01. (FIG. 28N) Schematic of the overall signaling pathway.

FIG. 29 illustrates knockdown efficiency of KLF2 and KLF4 in HUVECs as determined by quantitative RT-PCR. ***P<0.001 vs. control.

FIG. 30 illustrates expression levels of miR-335-5p and miR-335-3p in HUVECs. ***P<0.01.

FIG. 31 illustrates effect of miR-335 overexpression on mRNA levels of ICAM-1 and E-SELECTIN in HUVECs. **P<0.01 vs. control.

FIG. 32 illustrates infection efficiency of NF-κB reporter lentivirus in HUVECs. Transducted cells have constitutively expressed DsRed. Blue: Non-transduced HUVECs, Red: NF-κB reporter lentivirus transduced HUVECs.

FIG. 33 illustrates relative expression levels of NF-κB transcriptional complex and regulators in response to miR-335 overexpression in HUVECs.

FIG. 34 illustrates predicted alignment of miR-335-5p with human and mouse CHFR 3′ UTR.

FIG. 35 illustrates knockdown efficiency of CHFR in HUVECs by quantitative RT-PCR (left) and western blot (right). ***P<0.001 vs. control.

FIG. 36 illustrates relative expression levels of miR-335 in various tissues of Mir335−/− mice vs. controls. Skeletal muscle (Sk. Musc.) and brown adipose tissue (BAT) are shown. * P<0.05.

FIG. 37 illustrates food consumption of Mir335−/− mice compared to control littermates over 3 day period (n=8 per group).

FIGS. 38A-38B. illustrates gating strategy of the flowcytometry for CHFR-GFP and miR-335 co-transfection. (FIG. 38A) GFP expression in pCMV-GFP (left) and pEGFP-C2 CHFR transfected cells. Black box represents the cell population selected for further analyses of ICAM-1 and E-SELECTIN expression. (FIG. 38B) Percent GFP positive cell in each of the four conditions.

FIG. 39 is table showing the list microRNAs downregulated by statin stimulation (log 2 fold change<−0.25, P<0.05)

FIG. 40 is table showing the list microRNAs upregulated by statin stimulation (log 2 fold change>0.25, P<0.05)

FIGS. 41A-41M are tables showing the transcripts downregulated by miR-335 overexpression (log 2 fold change<0.25, P<0.07)

FIGS. 42A-42K are tables showing the transcripts upregulated by miR-335 overexpression (log 2 fold change>0.25, P<0.07)

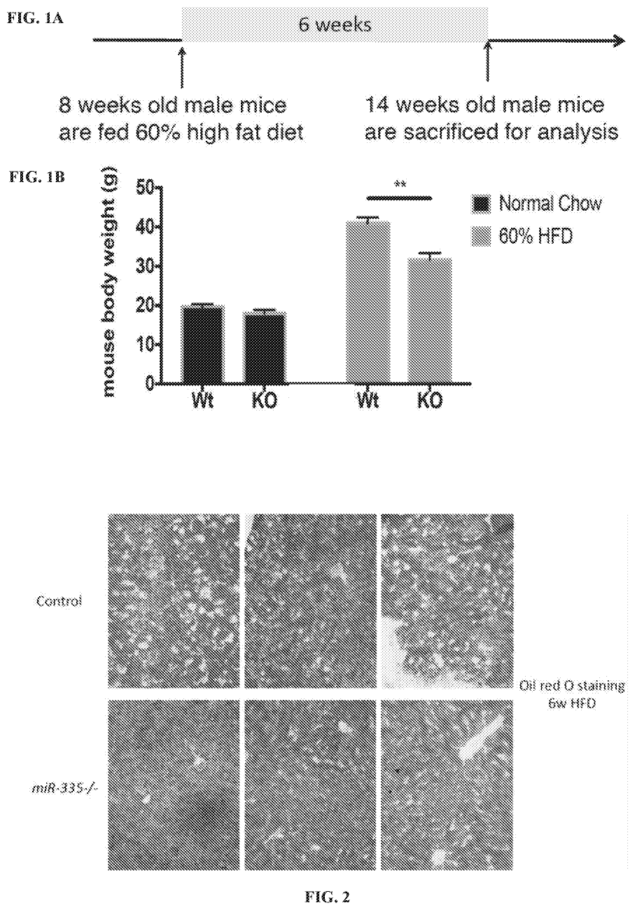

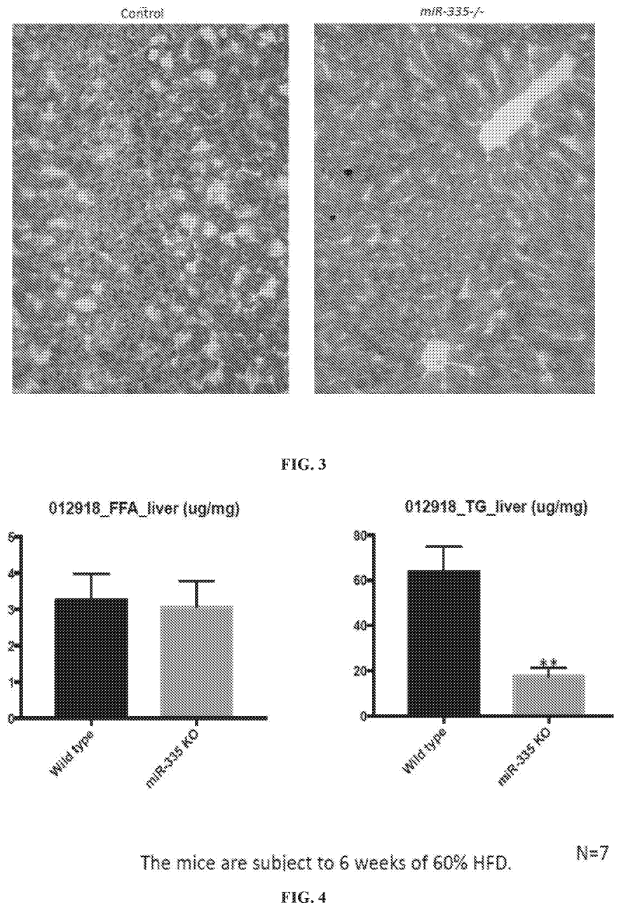

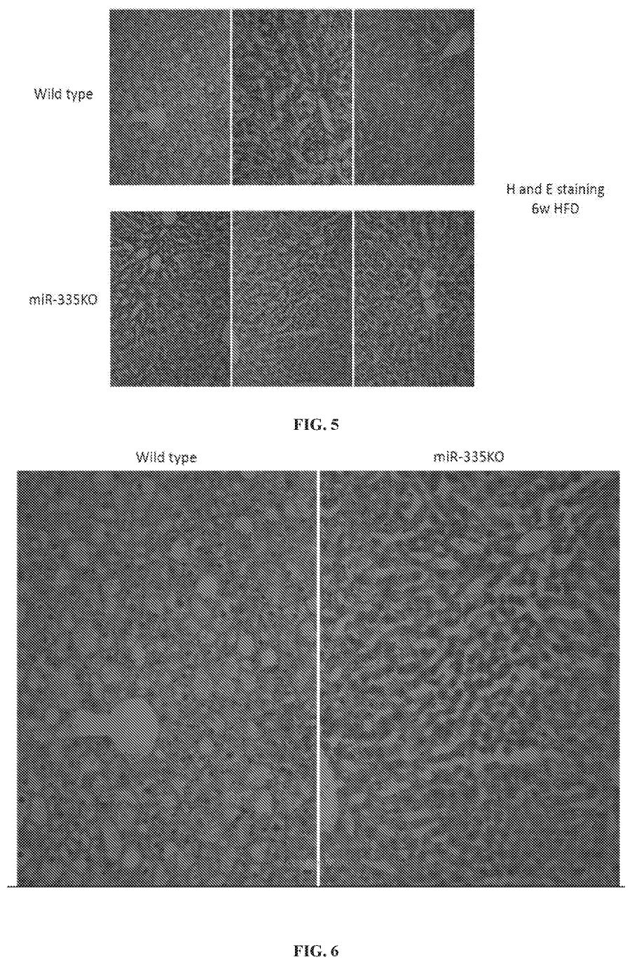

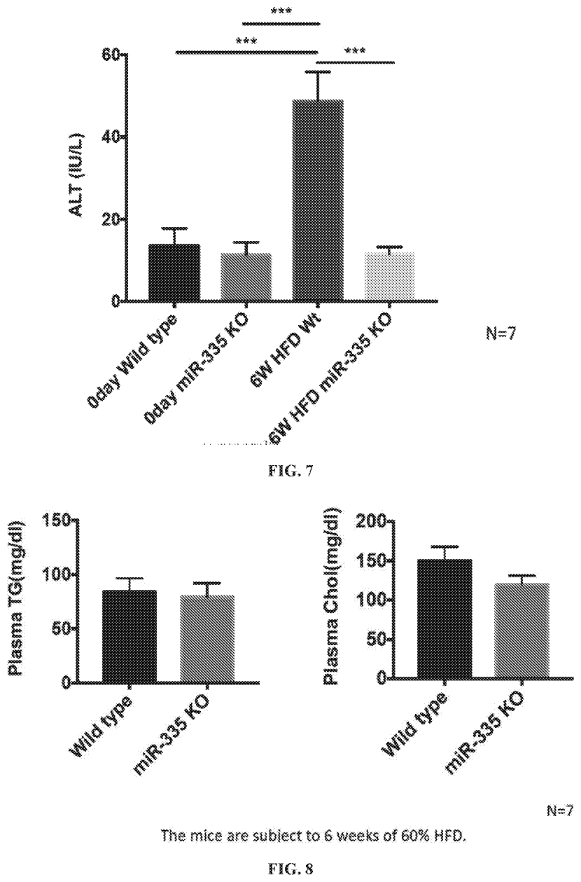

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, “microRNA 335”, “miRNA335” or “miR-335” are used interchangeably and refer to the microRNA encoded by the following sequence for the human homolog (SEQ ID NO:1):

TGTTTTGAGCGGGGGTCAAGAGCAATAACGAAAAATGTTTGTCATAAAC
CGTTTTTCATTATTGCTCCTGACCTCCTCTCATTTGCTATATTCA

As used herein, “microRNA 335 inhibitor”, “miRNA335 inhibitor” or “miR-335 inhibitor” are used interchangeably and refer to any molecule or technique that may be used to reduce the expression or activity of mir335. In various embodiments, the miR-335 inhibitor may be an antagomir targeting miR-335.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate: powdered tragacanth: malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol: polyols, such as glycerin, sorbitol, mannitol and polyethylene glycol: esters, such as ethyl oleate and ethyl laurate: agar; buffering agents, such as magnesium hydroxide and aluminum hydroxide; surface active agents; alginic acid; pyrogen-free water; isotonic saline: Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein. “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Methods of Treating Disease

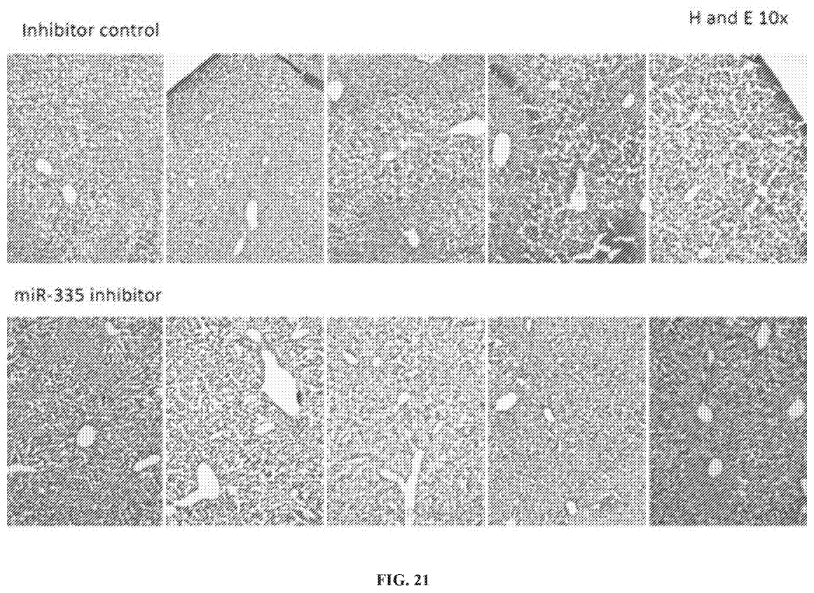

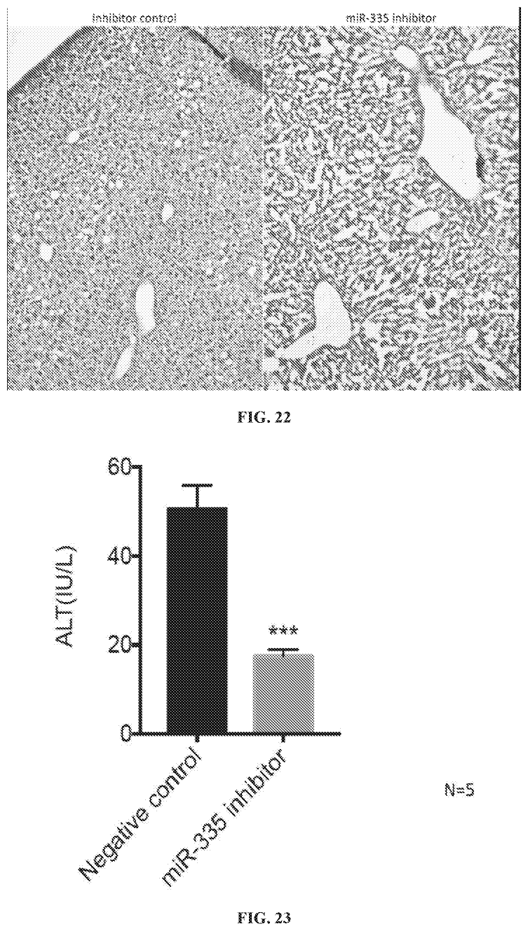

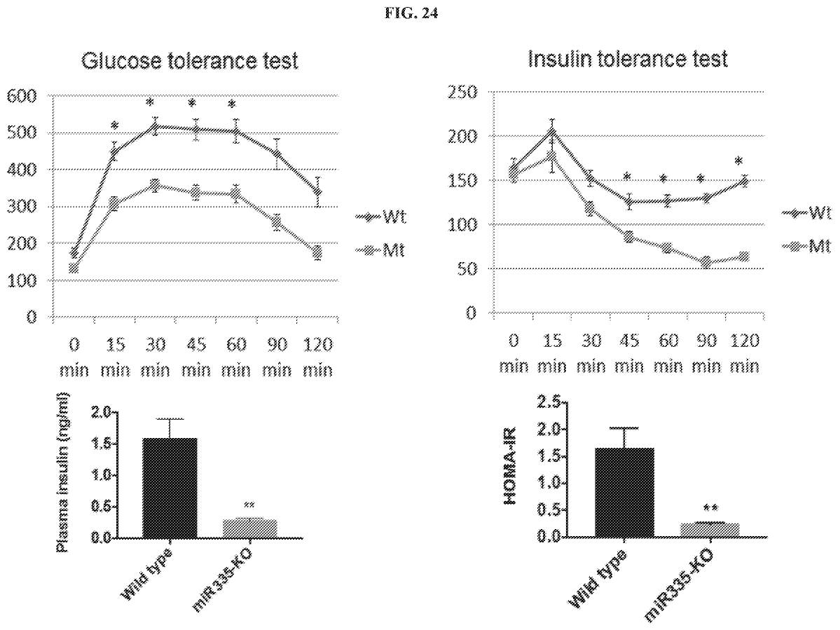

Without wishing to be limited by theory, the invention is derived in part from the discovery that inhibition of microRNA 335 can be applied to treat a subject for certain metabolic, cardiovascular, and/or hepatic disorders. In one aspect, the invention provides a method of treating non-alcoholic steatohepatitis (NASH) in a subject in need thereof, the method comprising administering to the subject a therapeutically effective amount of at least one microRNA 335 inhibitor. FIGS. 21-22 show that liver histology is preserved in animals on a high fat diet taking miR335 inhibitors compared to control animals.

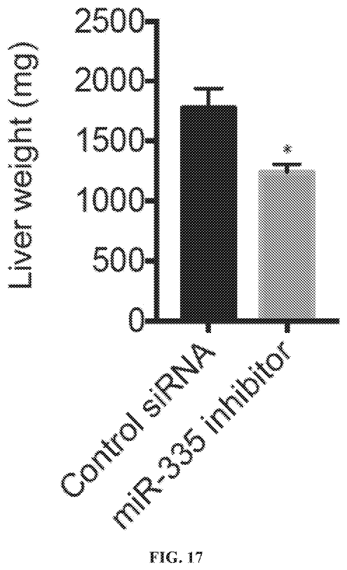

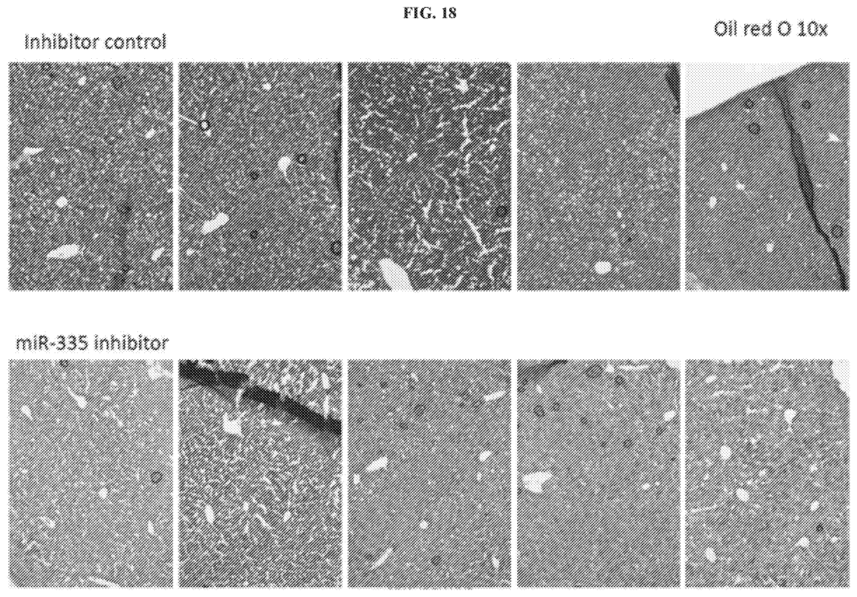

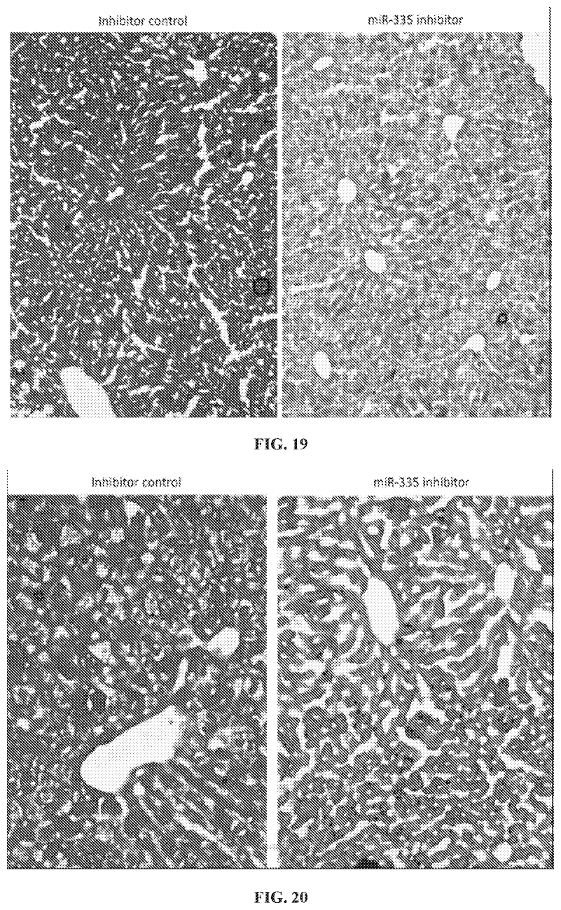

In another aspect, the invention provides a method of treating non-alcoholic fatty liver disease (NAFLD) in a subject in need thereof, the method comprising administering to the subject a therapeutically effective amount of at least one miRNA335 inhibitor. FIG. 17 shows that liver weight is decreased in animals taking miR335 inhibitors. FIGS. 18-20 show that fat accumulation in the liver is attenuated in animals taking miR335 inhibitors.

In another aspect, the invention provides a method of treating diabetes in a subject in need thereof, the method comprising administering to the subject a therapeutically effective amount of at least one miRNA335 inhibitor.

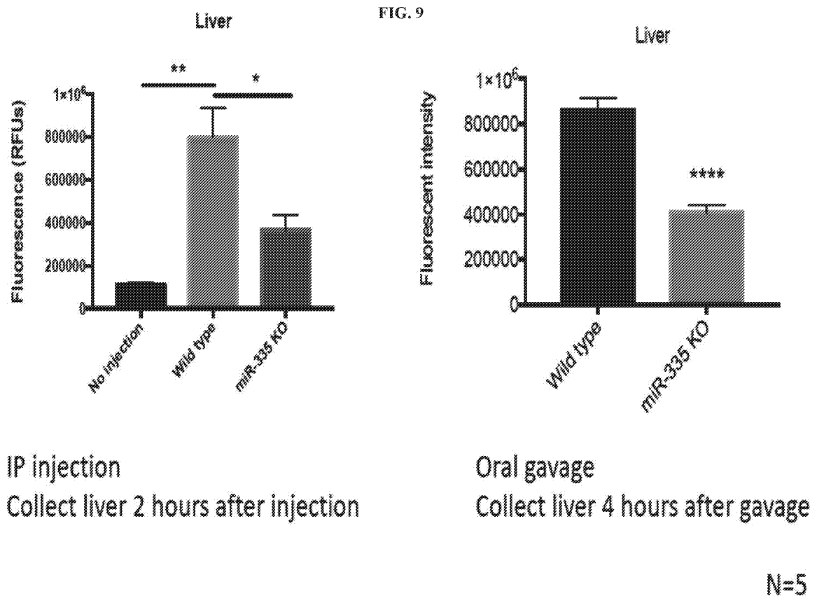

In another aspect, the invention provides a method of treating atherosclerosis in a subject in need thereof, the method comprising administering to the subject a therapeutically effective amount of at least one miRNA335 inhibitor. miR335−/− mice were protected from the atherosclerosis promoting effects of a high fat diet. FIG. 8 shows that plasma lipid profile is not changed in miR335−/− mice. FIG. 9 shows that fatty acid uptake into the liver is decreased in miR335−/− mice.

In another aspect, the invention provides a method of treating obesity in a subject in need thereof, the method comprising administering to the subject a therapeutically effective amount of at least one miRNA335 inhibitor. FIG. 16 shows that the weight gain associated with a high fat diet is suppressed in animals taking miR335 inhibitors compared to control animals.

In various embodiments, the at least one miRNA335 inhibitor comprises an antagomir. In various embodiments, the at least one miRNA335 inhibitor comprises a crispr cas9 mediated deletion of mir-335 in the liver or a virally transduced shRNA for mir-335. In various embodiments, the at least one miRNA335 inhibitor is formulated as a pharmaceutical composition further comprising at least one pharmaceutically acceptable carrier.

In various embodiments, the subject is a mammal. In various embodiments, the subject is a human.

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient: and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday. and so on.

In the case wherein the patient's status does improve, upon the doctor's discretion the administration of the inhibitor of the invention is optionally given continuously: alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%. 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples are here described:

### Mice

The mir335−/− mice and Apoe−/− mice have been previously described. All animals were maintained on a C57BL/6 background. For mice fed a high-fat diet, mice were administered a 60% fat diet (TD-06414, Envigo) or 40% fat diet with 1.25% added cholesterol (DI2108C, Research Diet) for designated times before tissue and serum were collected.

### Cell Culture and Reagents

Human umbilical vein endothelial cells (HUVECs) were cultured in EBM-2 (Lonza) and THP-1 monocytes (ATCC) were grown in RPM. Human coronary microvascular endothelial cells (HCMECs) (Lonza) were cultured in EBM (Lonza). For experimental treatments, HUVECs and HMVECs (passages 3 to 8) were grown to 70-90% confluence. Simvastatin (Sigma) was activated to its active form as previously described. Atorvastatin (Sigma) was solubilized in DMSO and used at 10 μM concentration. Transient transfection of plasmids was performed with Fugene HD (Promega) according to the manufacturer's instructions. For small interfering RNA (Invitrogen) and miR-335-5p mimic (Dharmacon) transfections, each oligonucleotide formulation and respective controls (control siRNA: 12935112 (Invitrogen), miRNA mimic control: 0010000105 (Dharmacon), 50 nM) were complexed with Lipofectamine RNAiMAX (Invitrogen) according to the manufacturer's instructions. For NF-κB inhibition, parthenolide (P0067, Sigma) was solubilized in DMSO and used at 5 nM concentration.

### RNA Extraction and Analyses

Total RNA was extracted with the miRNeasy Mini kit (Qiagen). Purified RNA was reverse transcribed with the iScript cDNA Synthesis Kit (Bio-Rad) and the TaqMan™ MicroRNA Reverse Transcription Kit (43665%, ThermoFisher). RT-PCR was performed with TaqMan probes for genes and miRNAs (ThermoFisher), or SYBR green assays for genes (Bio-Rad). All miRNA data were normalized to the internal control small RNAs U6 for human samples. For the mRNA samples, ribosomal 18S was used as an internal control. Individual RT-PCRs were performed on a CFX % (Bio-Rad) according to the manufacturer's instructions.

Gene and microRNA Expression Profiling Analyses

For miRNA array, HUVECs were treated with 10 μM of simvastatin for 24 h. RNA was extracted using miRNeasy Mini Kit (Qiagen). The RNA was quantified and the quality verified. RNA samples (200 ng) from each condition were first labeled and then hybridized to each array using standard Illumina protocols. Sample array matrices were scanned on an Illumina BeadArray reader. Data were imported into GenomeStudio (Illumina), quantile normalized and log 2 transformed in R (http://www.r-project.org/).

For gene expression profiling in response to miR-335 overexpression, HUVECs were transduced with human pre-miRNA expression lentivector for either miR-335 or a negative control (System Biosciences) for 72 h, after which RNA was extracted using miRNeasy Mini Kit (Qiagen). The RNA was quantified and the quality verified. The HumanHT-12 v4 Expression BeadChip Kit (Illumina) was used according to the manufacturer's protocol. Microarray results were analyzed using the bead array and limma packages in R/Bioconductor (v 2.14/2.09). Array data have been submitted and are publicly available at Gene Expression Omnibus (GEO).

### Flow Cytometry

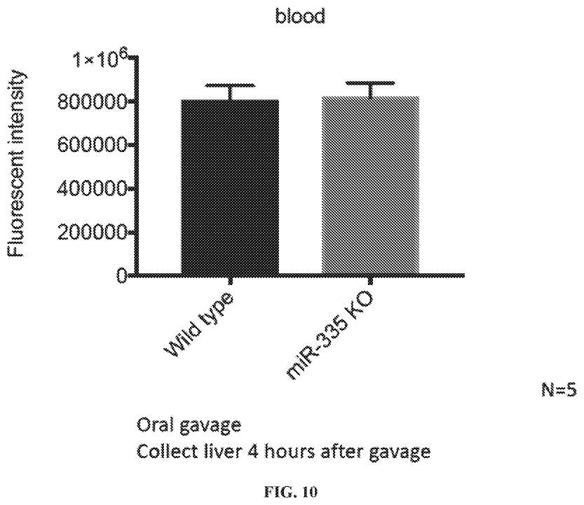

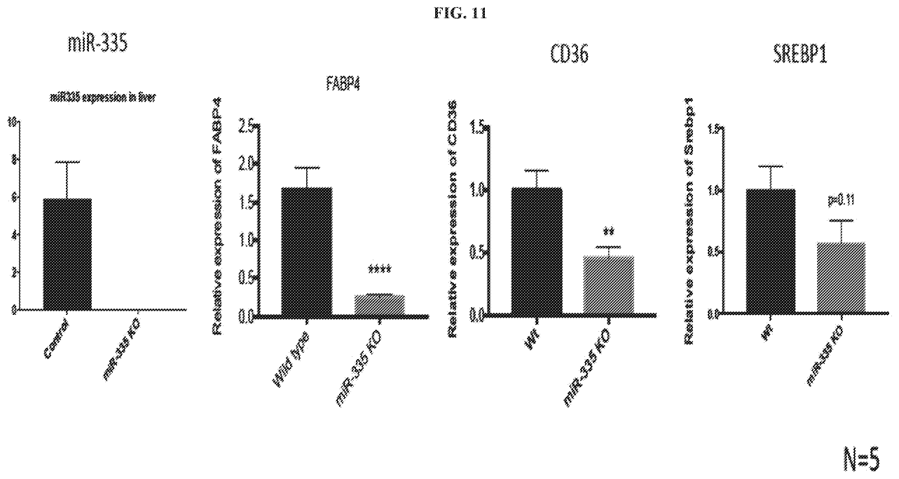

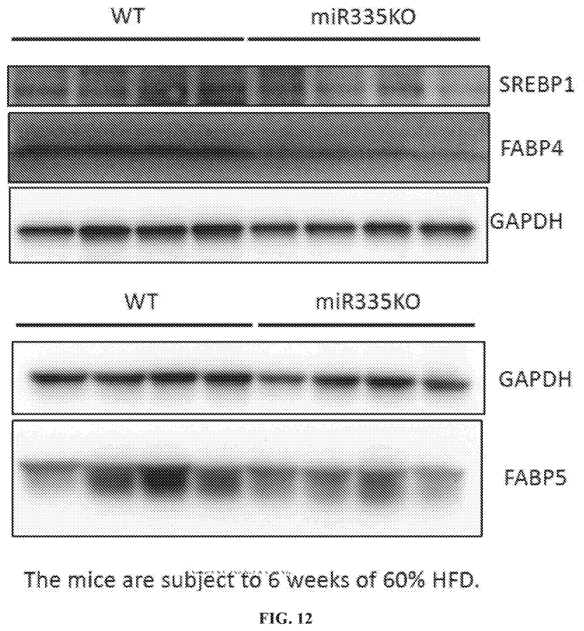

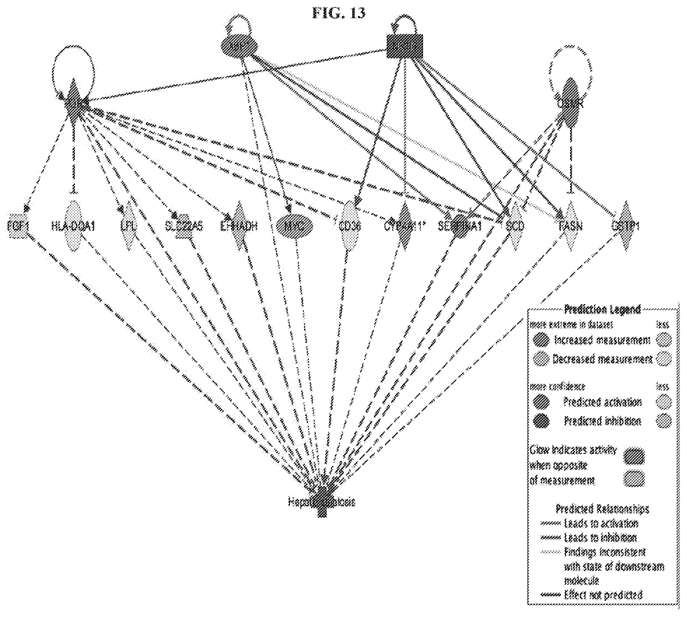

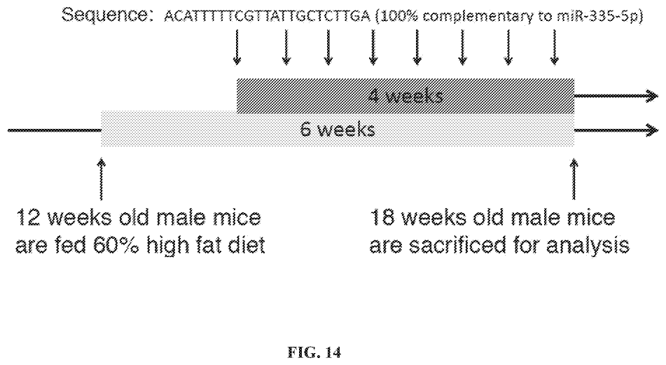

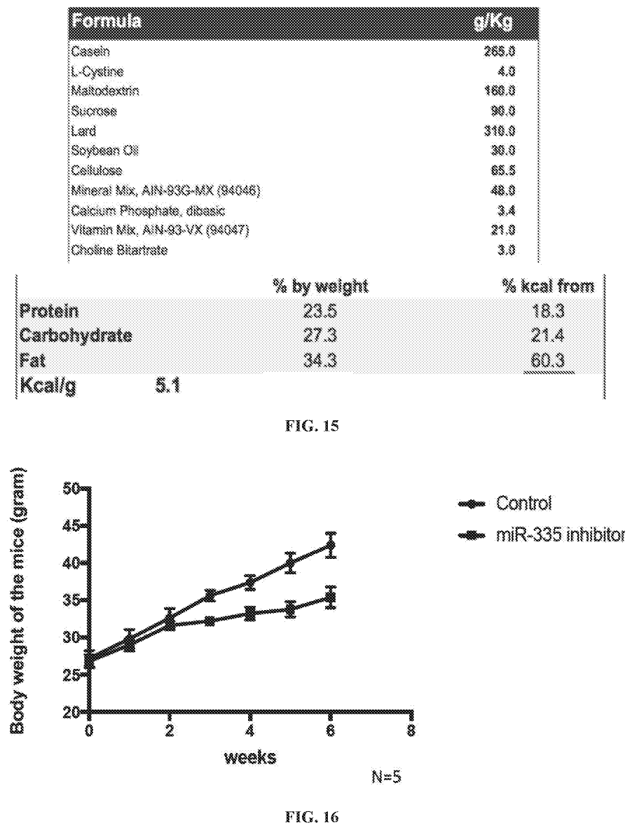

Transfected cells were stained with PE conjugated anti-CD62E (E-SELECTIN) antibody (551145, BD Biosciences) and APC-conjugated anti-CD54 (ICAM-1) antibody (559771, BD Biosciences). For CHFR rescue studies, HUVECs were transfected with pEGFP-C2 CHFR (61853, Addgene) or pCMV-GFP (11153, Addgene) (1 μg) using FugeneHD (E2312, Promega). Either miR-335 or negative control mimic (50 nM) were transfected 24 h after using RNAiMAX (Invitrogen). Cells were stained and gated based on GFP expression to restrict our analyses to those cells that were transfected with the respective plasmids (FIGS. 10A-10B). Flow cytometry was performed using a LSRII flow cytometer (BD Biosciences). Data quantification was performed with FlowJo 10.6 software.

### Monocyte Adhesion Assay

Endothelial cells (ECs) were transfected with control or miR-335 mimic, and also transfected with control or CHFR siRNA for 72 h. THP-1 monocytes labeled fluorescently (BCECF-AM, B1150, Invitrogen) were incubated with the ECs for 1 h at 37° C. Following removal of non-adherent cells by washing, monocyte adhesion to HUVECs was quantified by measuring the fluorescent cell areas (Adobe Photoshop CS4) in five independent images per condition. The graph depicts average of three independent experiments.

### Luciferase Assays

For determination of NF-κB luciferase reporter activity. HUVECs were transfected with pGL4.32[luc2P/NF-κB-RE/Hygro] luciferase reporter vector (Promega) in combination with Renilla luciferase vector for normalization with FugeneHD (E2312, Promega). Cells were then transfected with miR-335 or control mimic (50 nM) and lysed in lysis buffer (Promega). Dual Luciferase Reporter System (Promega) was used according to the manufacturer's protocol. For 3′ UTR analysis, human CHFR 3′ UTR reporter construct was used (SwitchGear Genomics). For mutation analysis, the sequence 5′-GCTCTTGA-3′ in the predicted miR-335-5p binding site was deleted. HUVECs were transfected with the luciferase reporter constructs containing the CHFR 3′ UTR variants using FugeneHD (E2312, Promega), and subsequently with either miR-335 mimic or negative control miRNA (50 nM) with RNAiMax (Invitrogen) according to the manufacturer's instructions. Luciferase activity was measured using the LightSwtich assay kit (SwitchGear Genomics) according to the manufacturer's instructions. All experiments were performed three times in triplicates.

### Viral Infection

Lentiviral NF-κB-GFP reporter plasmids were used. A dual-color, lentiviral NF-κB fluorescence reporter system was generated using a modified pCS vector. Three tandem NF-κB response elements (GGGAATFTCCC (SEQ ID NO: 2)) were inserted before a minimal CMV promoter and d2EGFP (GFP with a PEST sequence, conferring a ˜2 h half-life). This vector additionally encodes a PGK promoter-driven DsRed, as an internal reference. A beta globin insulator was inserted between the two cassettes. The plasmid was co-transfected with pVSVG and psPAX2 packaging plasmids into HEK-293T cells with Lipofectamine 2000 (Life Technologies). Viral supernatants were collected and concentrated with Lenti-X Concentrator (Clontech).

### Western Blot

Western blots were conducted as previously described. Cells were harvested in RIPA buffer (Thermo Fisher Scientific) supplemented with protease and phosphatase inhibitors (Thermo Fisher Scientific). Protein concentration was measured using Bradford reagent (BioRad). Mouse samples were dissected, washed in PBS and snap-frozen in liquid nitrogen and homogenized in RIPA buffer. The primary antibodies used were: CHFR (6904, Cell Signaling); FABP4 (AF1443, R&D Systems); GAPDH (2118, Cell Signaling).

### In Situ Hybridization

In situ hybridization was performed in close cooperation with Bioneer.24 Double-digoxigenin-labeled locked nucleic acid (LNA) probes specific for miR-335-5p ACATTTTTCGTTATTGCTCTTGA (SEQ ID NO:3), predicted RNA Tm:84° C.), miR-126-3p as positive control (CATTATTACTCACGGTACGA (SEQ ID NO:4)), predicted RNA Tm: 92° C.), and a scrambled probe used as a negative control (TGTAACACGTCTATACGCCCA (SEQ ID NO: 5)), predicted RNA Tm: 87° C.) were custom designed from Exiqon (Qiagen). The scrambled probe consists of a random sequence with no known complementary sequence target among human transcripts. Briefly, the probes were incubated on 5 μm-thick paraffin sections at 10-20 nM in Exiqon ISH buffer (Exiqon) at 55-60° C. The probes were detected with alkaline phosphatase-conjugated anti-DIG antibodies (Merck) and incubated in 4-nitroblue tetrazolium (NBT) and 5-bromo-4-chloro-3′-indolylphosphate (BCIP) substrate (Merck) for 2 h. The sections were counterstained with Nuclear Fast Red (Vector Laboratories), and dehydrated in ethanol dilutions and mounted with Eukitt Mounting Medium (VWR). Images were acquired using Nikon 80i light microscope.

### Aortic Plaque Lipid Quantification

Mice were euthanized after 16 weeks of being fed a high-fat diet. Aortas were fixed overnight at 4° C. in 4% paraformaldehyde in PBS, the dissected luminal surface was exposed, and pinned to a silicon plate. Aortas were washed in PBS and then dehydrated using propylene glycol for 2 min at room temperature. Aortas were stained with 0.5% Oil Red 0 (01391, Sigma) in isopropanol for 15 min at room temperature, then washed with 85% propylene glycol, followed by PBS. The percent area stained by Oil Red 0 was quantified using ImageJ (NIH). Aortic valves were stained with H&E to analyze the aortic valve plaque area and valve thickness. Quantifications were made using ImageJ.

### Immunohistochemistry

Immunohistochemistry was conducted as previously described. The primary antibodies used were: anti-mouse CD31 (553370, BD Pharmingen), FABP4 (AF1443, R&D systems), CHFR (6904, Cell Signaling), ICAM-1 (A5597, ABclonal) and E-SELECTIN (A2191, ABclonal). The secondary antibodies used were: Alexa Fluor 488 (A11006, Invitrogen), 594 (A11011 and 11057, Invitrogen). Samples were imaged with fluorescent microscopy (Nikon 80i).

### Intraperitoneal Glucose/Insulin Tolerance Tests

Glucose tolerance and insulin tolerance tests were conducted as previously described, and data collected at designated times after glucose or insulin administration.

### Plasma Insulin Analysis

Plasma was assayed for insulin (90080, Crystal Chem) according to the manufacturer's protocol. Briefly, fresh plasma samples were collected by retro-orbital blood collection. The samples were diluted into dilution buffer and added to antibody-precoated 96 well plates. After the plates were incubated and washed, conjugate solution, substrate solution, and stop solution were subsequently added and OD at 450/630 nm was measured using a microplate reader (Bio-Tek).

### Fatty Acid Uptake and Permeability Assay

Fatty acid uptake and permeability assays were conducted as previously described. In brief, HUVECs were plated onto 24-well plates and pre-treated with 50 nM of miR-335 mimic or CHFR siRNA for 72 h before being incubated with 2 μM BODIPY 558/568 C12 (BODIPY) (D3823, Life Technologies) for 90 min. Cells were washed and fluorescence was measured using a fluorescence plate reader (Bio-Tek). For the Transwell fatty acid transport assay. HUVECs were transfected with miR-335 mimic or CHFR siRNA and their respective controls, and plated on Transwell inserts (0.4 μM pore size; 3413, Costar) in 24-well plates and cultured for 72 h. Fluorescence was measured from the bottom chamber at 0, 1, 2, 4 and 6 h.

### Oil Red O Staining in Skeletal Muscle

Samples were sliced into 10 μm-thick sections and then fixed with 4% paraformaldehyde for 5 min. Sections were incubated with Oil Red 0 (01391, Sigma) for 10 min. After rinsing with 60% isopropanol and PBS, sections were mounted with aqueous mounting media. Images were obtained with a light microscope (Nikon 80i) and quantified using ImageJ.

### Tissue Lipid Analysis

Tissue lipid analysis was conducted as previously described. In brief, lipids were extracted using the method of Folch-Lees. Individual lipid classes were separated by thin layer chromatography and visualized by rhodamine 6G. Fatty acids were extracted and analyzed by gas chromatography. Gas chromatographic analyses were carried out on an HP 5890 gas chromatograph equipped with flame ionization detectors, an Agilent 7890A GC System, and a capillary column (SP2380, 0.25 mm×30 m, 0.25 μm film, Supelco, Bellefonte, Pa.).

### In Vivo Fatty Acid Uptake Assay

Mice (12 to 16 weeks old males) were given a bolus dose of 500 μM of BODIPY (D3823, Life Technologies) dissolved in 1% BSA/PBS by oral gavage. Organs were harvested 6 h after BODIPY administration. Tissues were collected (300 mg) and homogenized into 500 μl of RIPA buffer. After centrifuging the samples at 10,000 g for 10 min, 300 μL of supernatant was collected and dissolved into 900 μL of Dole reagent. After vortexing, the mixture was centrifuged at 18,000 g for 10 min. The supernatant was collected and the fluorescence was measured by a microplate reader (Bio-Tek).

### H&E Staining

Frozen 10-μm cryosections from the liver were washed with distilled water for 10 min. After the washing, sections were put into Eosin solution for 2 min. After rinsing with distilled water, sections were put into Hematoxylin solution for 5 min. After washing with distilled water for 15 min, the slides were mounted with DPX mountant (Sigma). Sections were imaged using bright-field microscopy.

### Plasma ALT Analysis

Plasma was assessed for alanine aminotransferase (ALT) (Pointe Scientific) following the manufacturer's instructions. Briefly, 20 μl of each plasma sample was added in the 200 μl of the reagent and incubated at 37° C. After 1 min incubation, samples were put on the plate and the plate was read at 340 nm with a microplate reader (BioTek Synergy 2). The ALT values were calculated according to the manufacturer's instructions.

### Statistics

All experiments were performed in triplicate (unless otherwise specified) in at least three independent experiments, and data shown are mean±S.E.M. When only 2 groups were compared, statistical differences were assessed with unpaired. 2-tailed Student's t test. Otherwise statistical significance was determined using 1- or 2-way ANOVA followed by Bonferroni multiple comparison test. A P value less than 0.05 was considered statistically significant.

### Example 1

### Determine Whether Statins can Induce Differential miRNA Expression in ECs

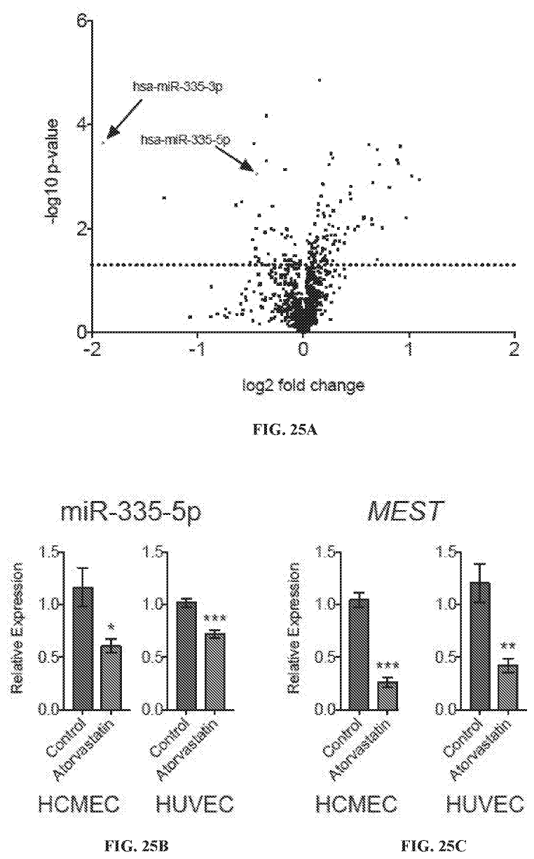

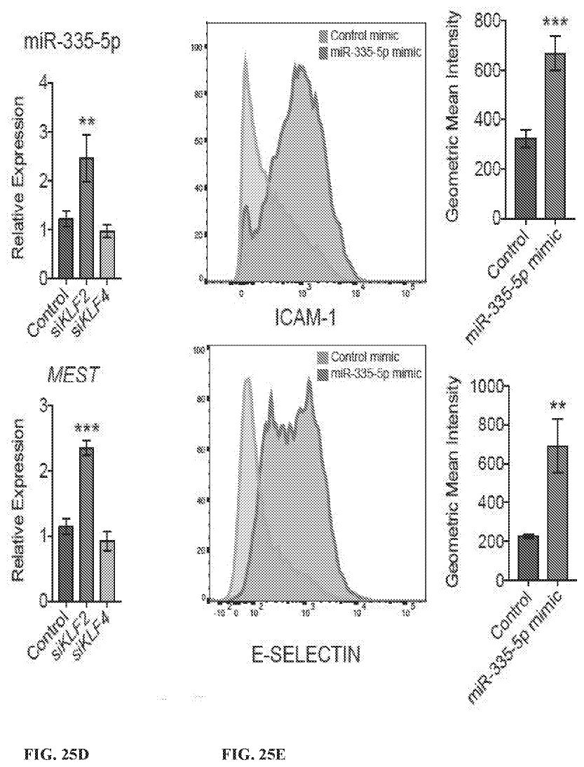

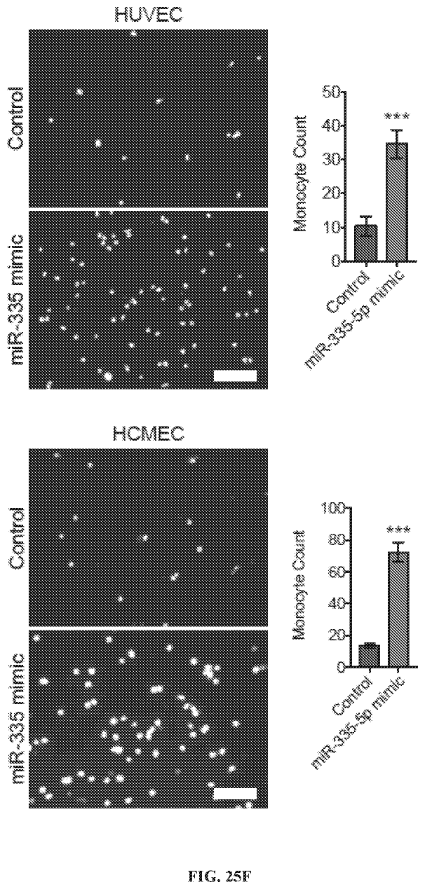

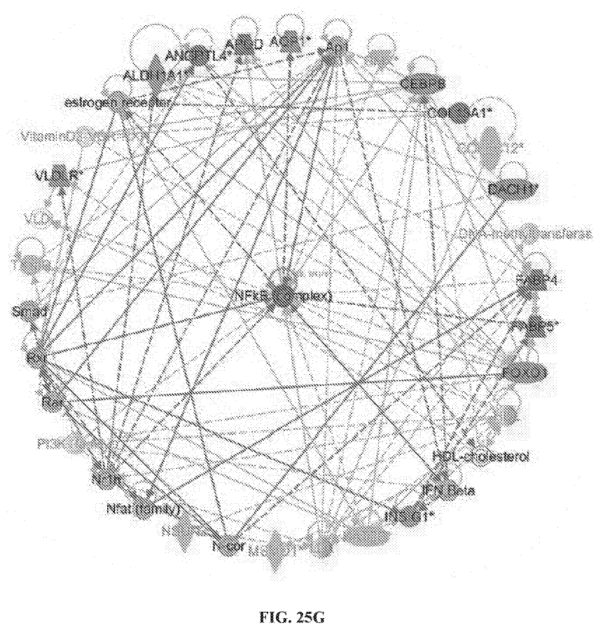

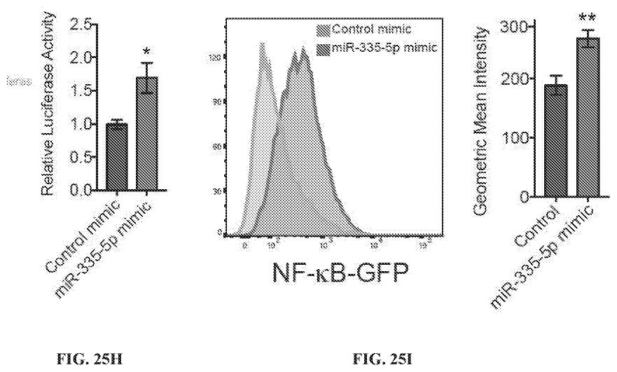

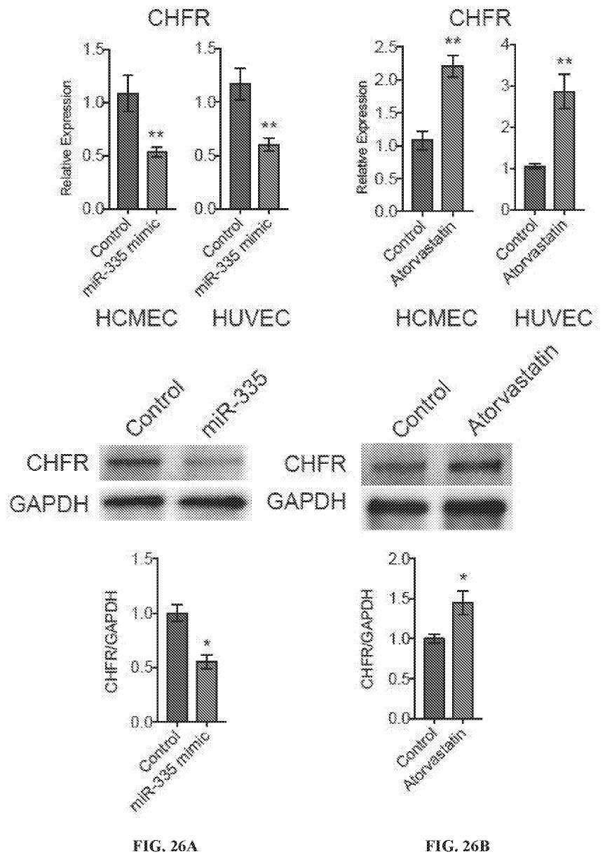

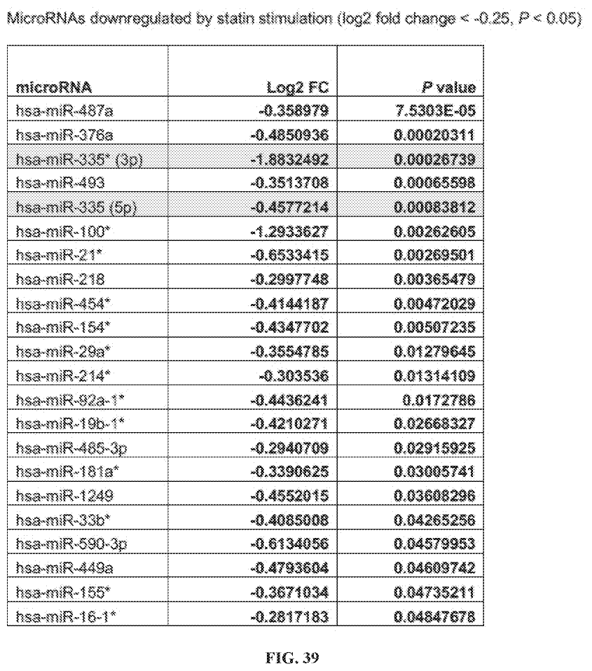

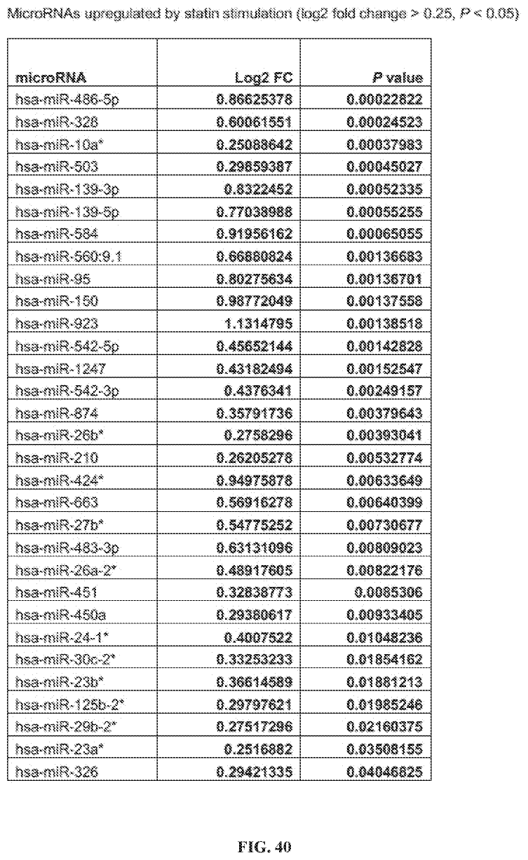

To determine whether statins can induce differential miRNA expression in ECs, miRNA expression profiling analyses was conducted using human umbilical vein endothelial cells (HUVECs) subjected to treatment with simvastatin. Of those miRNAs that were differentially regulated, miR-335-5p and miR-335-3p were two of the most significantly downregulated miRNAs in this analyses (FIGS. 25A, 39, 40). It was found that treatment with atorvastatin also led to significant decrease in miR-335 expression in HUVECs as well as human coronary artery microvascular endothelial cells (HCMECs) (FIG. 25B), suggesting a drug class effect of statins in regulating miR-335. As miR-335 is intronic to the gene MEST, it was confirmed that MEST itself is also significantly downregulated in response to atorvastatin treatment of HCMECs and HUVECs (FIG. 25C).

### Example 2

### Elucidating the Mechanism of miR-335 Regulation by Statins

It was sought to further elucidate the mechanism of miR-335 regulation by statins. Previous gene expression profiling analyses of cardiac microvascular ECs from mice with endothelial specific deletion of Klf2 and Klf4 had identified the miR-335 host gene Mest as the second most significantly upregulated gene. As endothelial KLF2 and KLF4 are known to be upregulated by statin treatment, it was determined whether modulation of KLF2 or KLF4 can affect miR-335 and MEST expression in ECs. It was found that siRNA mediated knockdown of KLF2, but interestingly not KLF4, led to significant increase in both MEST and miR-335 expression (FIGS. 25D, 29), suggesting that expression levels of miR-335 and MEST are repressed by KLF2.

### Example 3

### Characterizing the Function of miR-335 in ECs

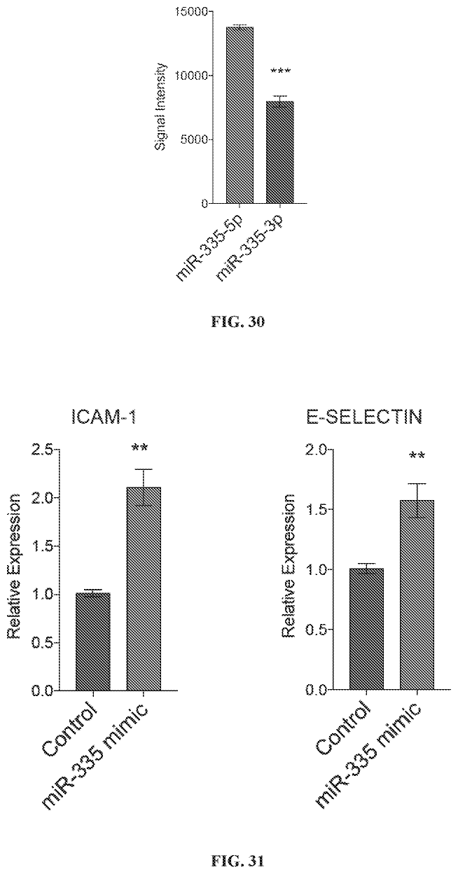

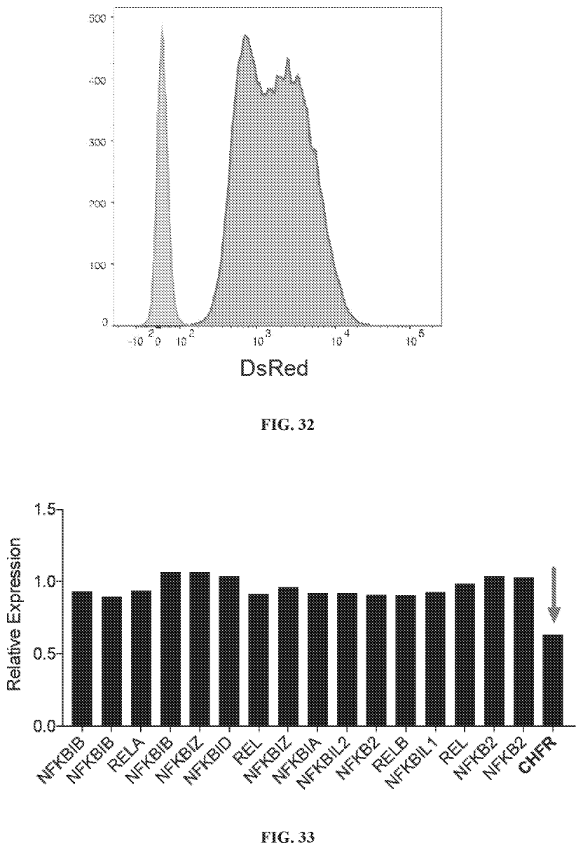

Overexpression of miR-335-5p mimic in HUVECs (miR-335-5p was expressed at a significantly higher level than miR-335-3p in ECs (FIG. 30) led to significantly increased expression of endothelial inflammatory markers, including ICAM-1 and E-SELECTIN (FIGS. 25E, 31). Moreover, overexpression of miR-335-3p had minimal effect on ICAM-1 and E-SELECTIN expression, hence the focus was on the effects of miR-335-5p (henceforth called miR-335) for overexpression studies. It was also found that monocyte adhesion to either HUVECs or HCMECs overexpressing miR-335-5p was significantly greater than ECs transfected with control mimics (FIG. 25F). These findings suggested that miR-335 has an important role in induction of endothelial inflammatory milieu.

### Example 4

### Defining Downstream Signaling Pathways Involved in the Pro-Inflammatory Cellular Response to Increased miR-335 Expression

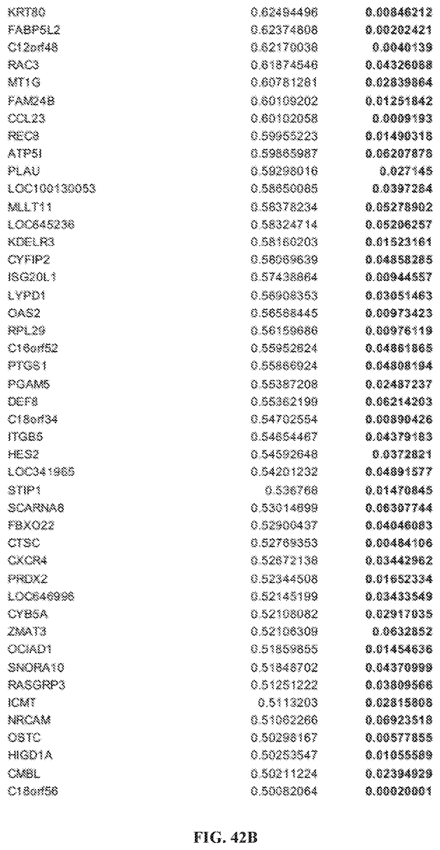

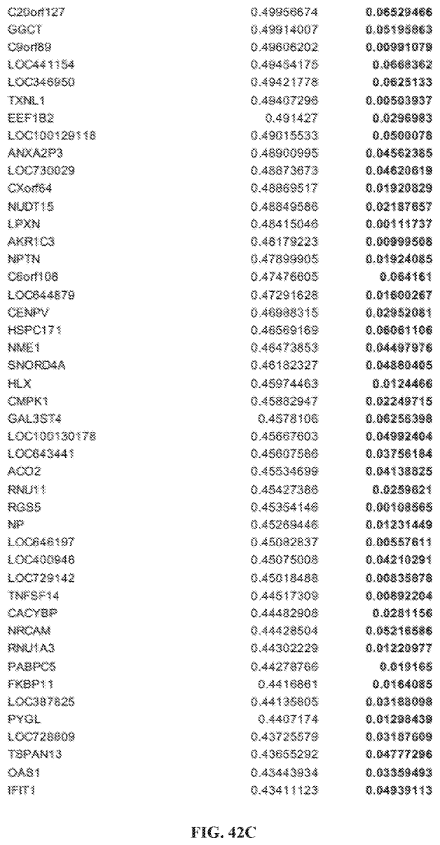

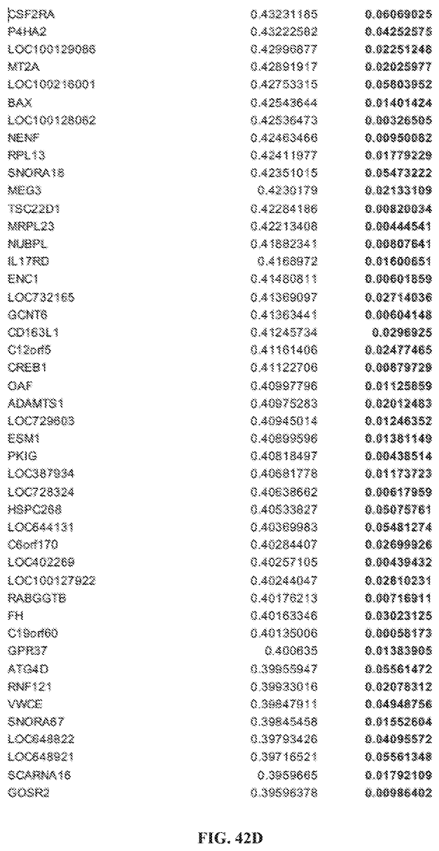

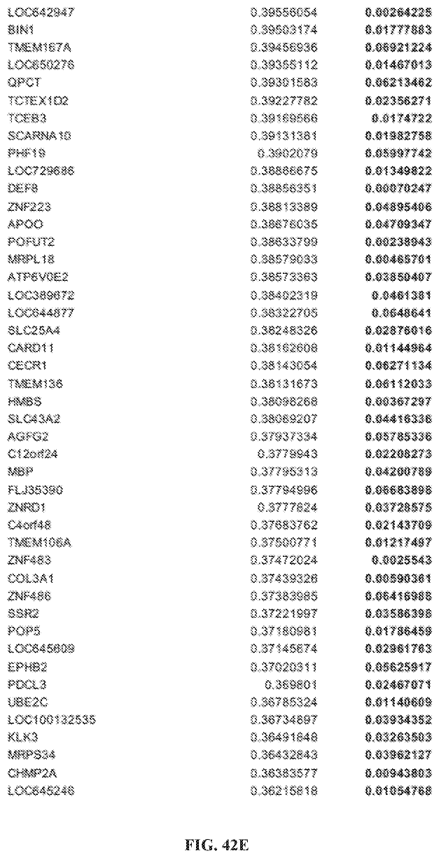

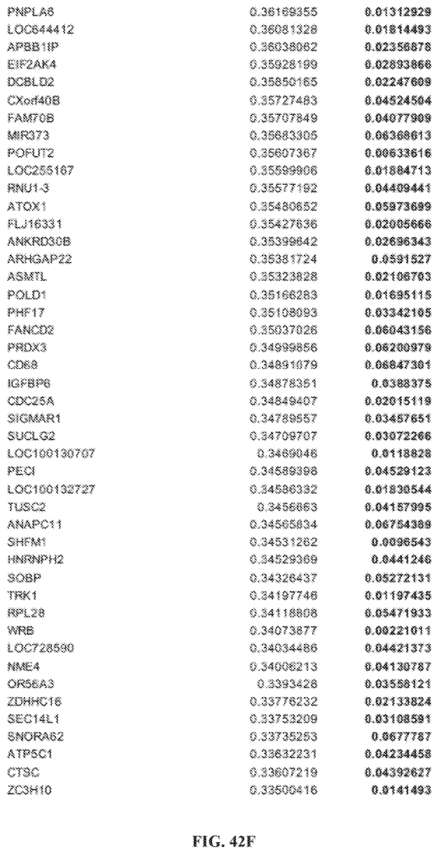

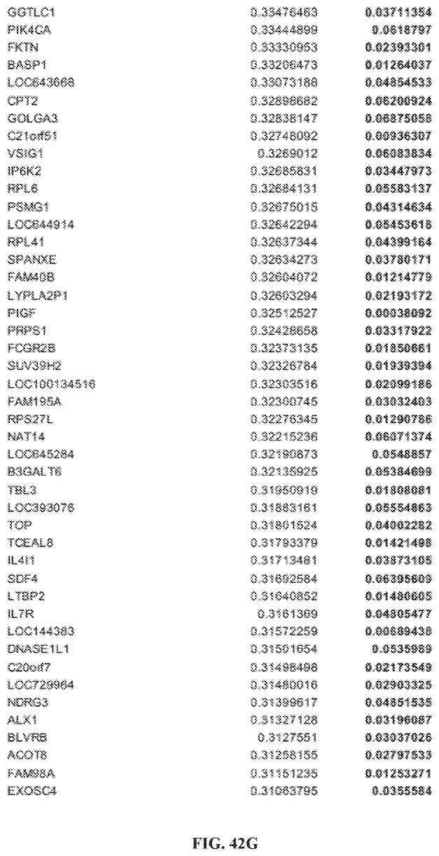

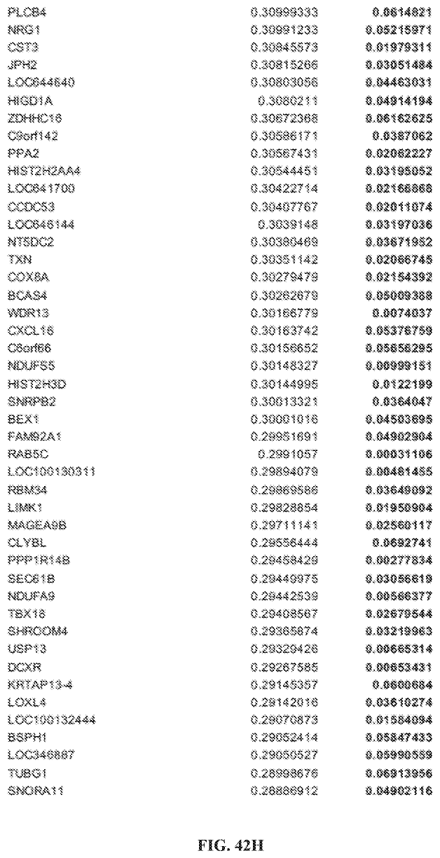

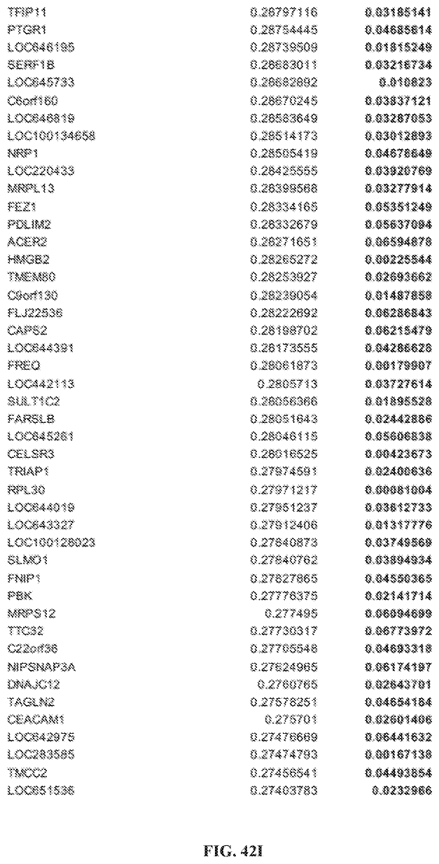

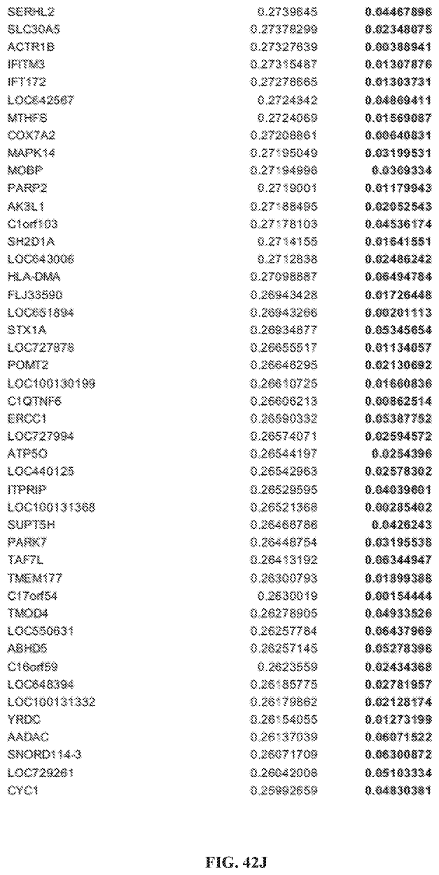

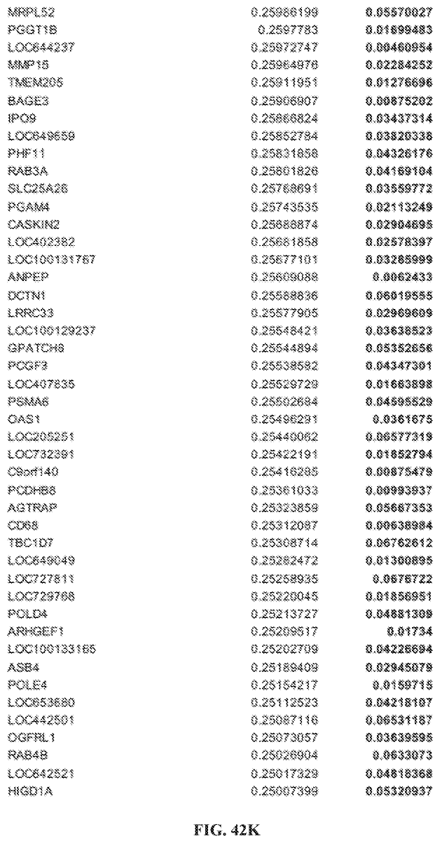

To further define the downstream signaling pathways involved in the pro-inflammatory cellular response to increased miR-335 expression, gene expression profiling analyses of HUVECs subjected to miR-335 overexpression was carried out. HUVECs were transduced with lentivirus overexpressing miR-335 or vector control, and gene expression profiling analyses were conducted. Ingenuity Pathway Analysis (IPA) of the data, restricted to genes with a relative 1.5-fold change of expression, generated eleven networks. The highest scoring network (i.e., network with the lowest probability of finding the molecules included in the network by chance) involved lipid metabolism and transport (FIG. 25G). Within this network, NF-κB was identified as a central node, with multiple downstream targets identified from the gene expression profiling dataset including a number of key metabolic proteins, including FABP4 (fatty acid binding protein 4) and FABP5 (FIGS. 25G, 41A-41M, 42A-42K). Next investigated whether miR-335, either directly or indirectly, activates NF-κB. First, it was found that miR-335 overexpression in HUVECs resulted in activation of NF-κB luciferase reporter (FIG. 25H). It was further validated the induction of NF-κB by miR-335 by using HUVECs stably transduced with a lentiviral construct containing both a green fluorescent protein (GFP) reporter driven by NF-κB responsive promoter (NF-κB-GFP reporter), and a DsRed that is expressed under the control of a constitutively active promoter (FIG. 42A-42K). It was found that there is a significant increase in GFP expression when these cells were transfected with miR-335 (FIG. 25I). Overall, these findings demonstrate miR-335 dependent activation of NF-κB, which in turn may be a key driver of the endothelial inflammatory response.

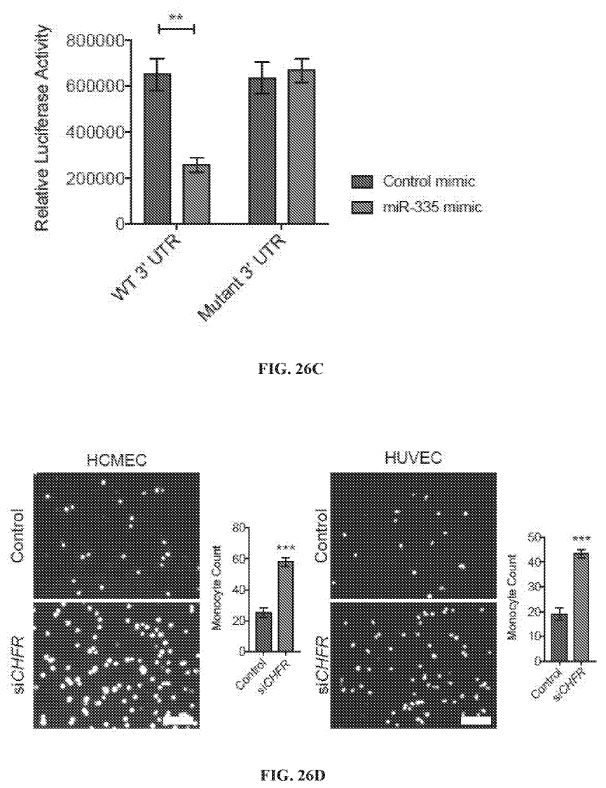

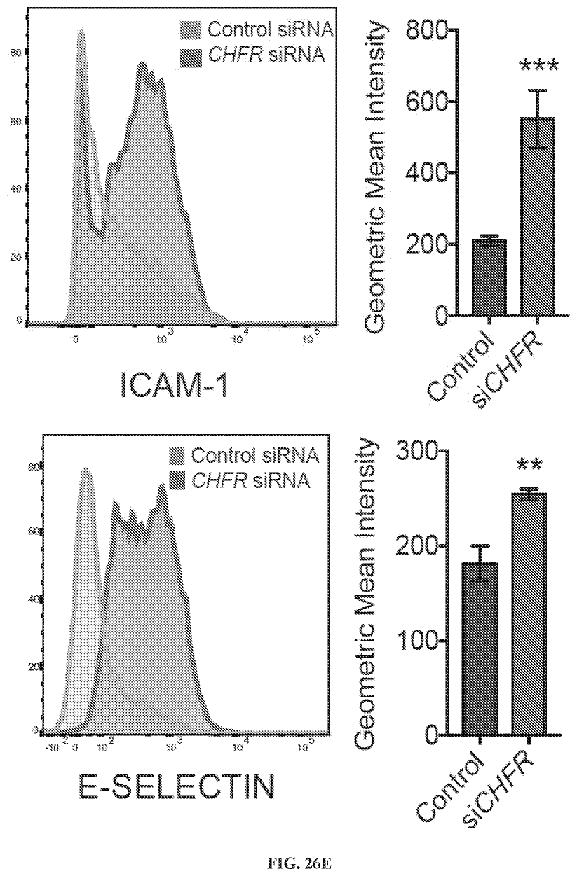

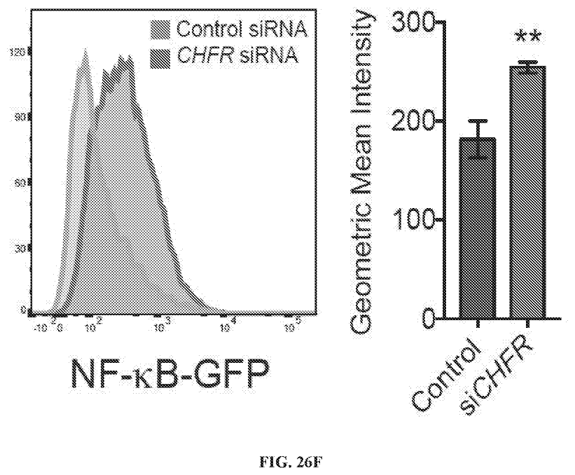

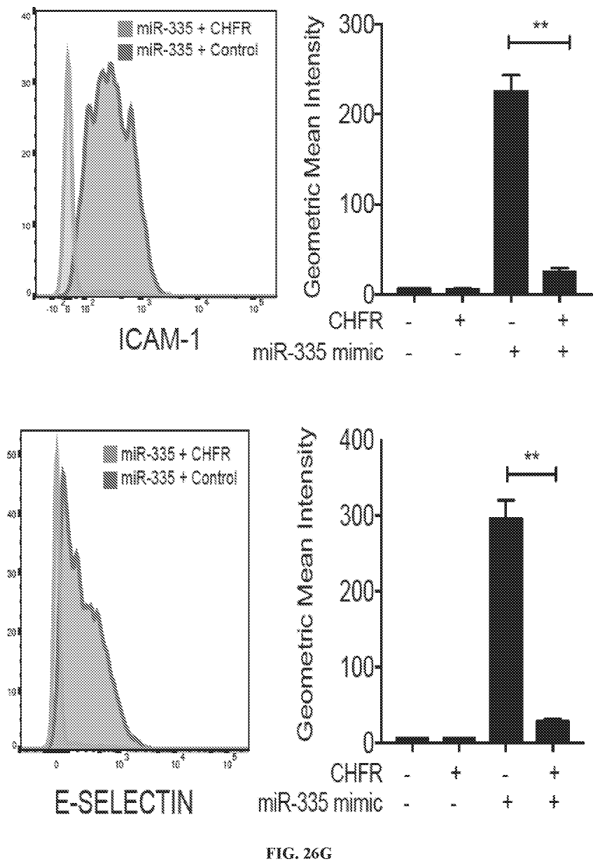

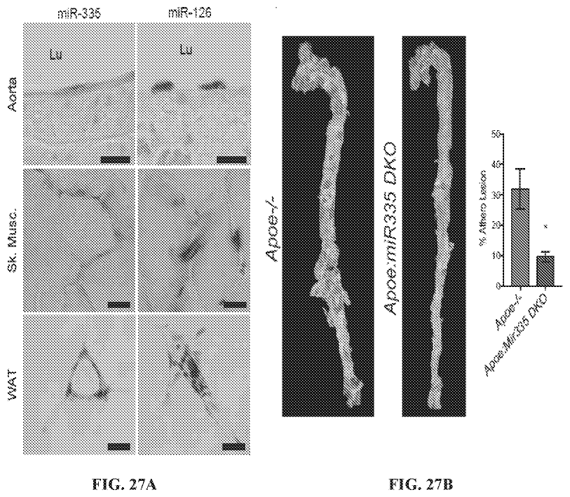

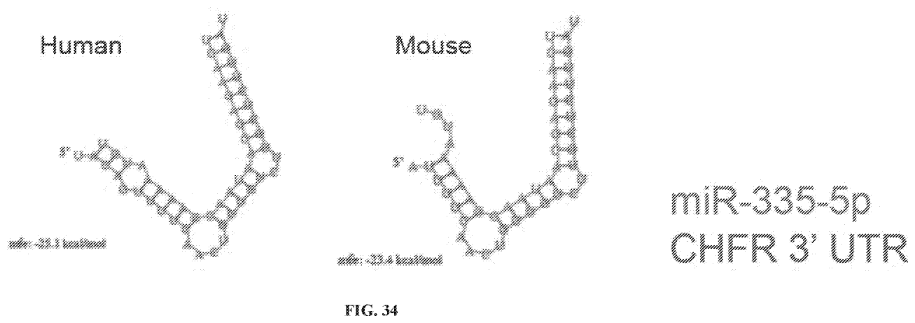

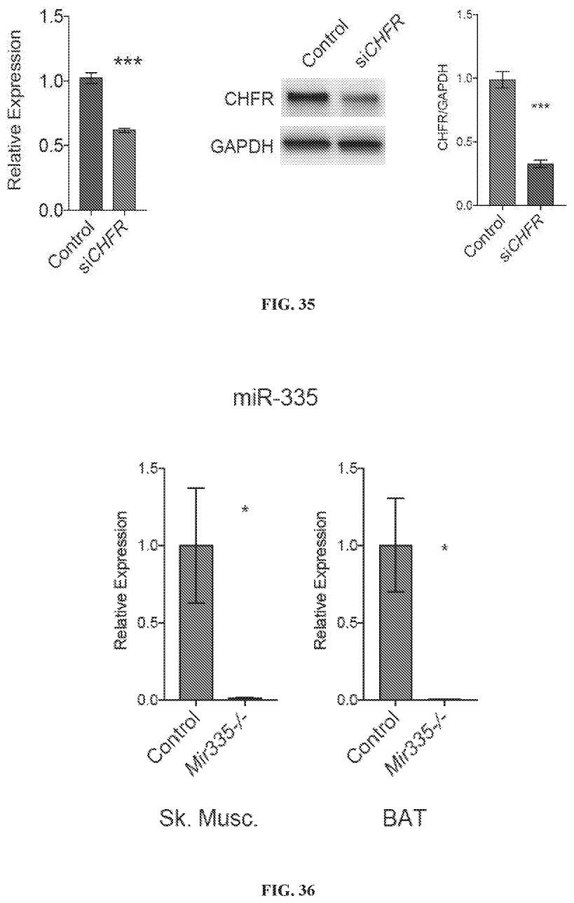

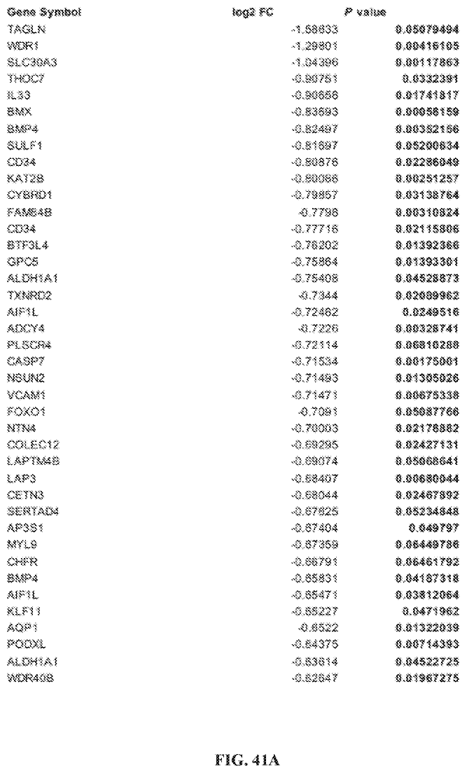

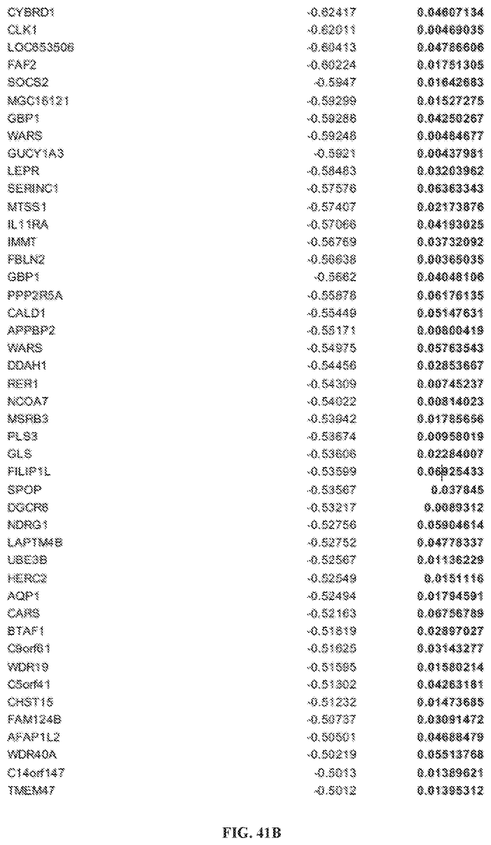

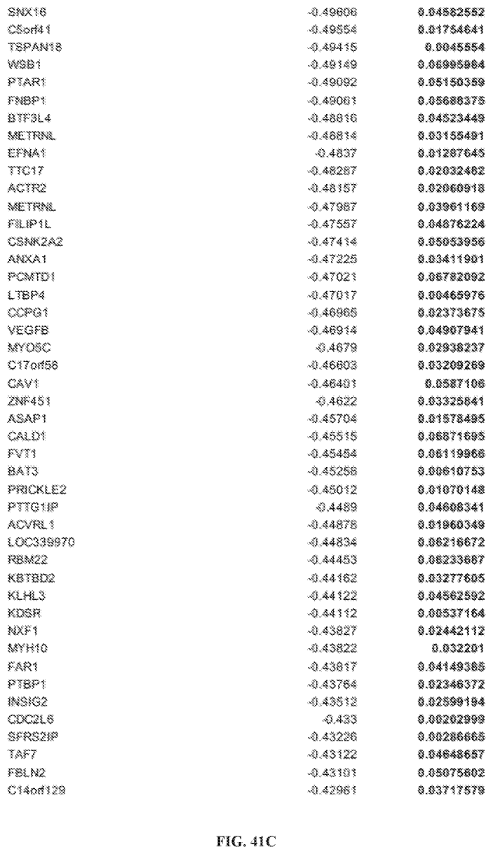

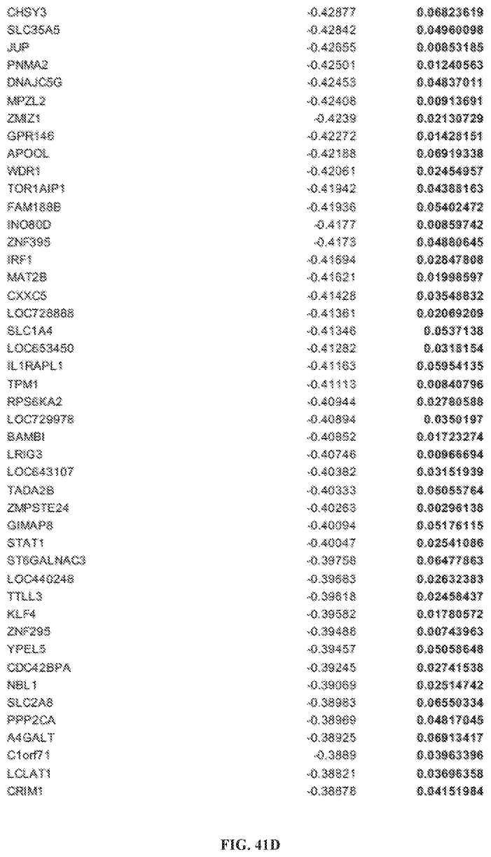

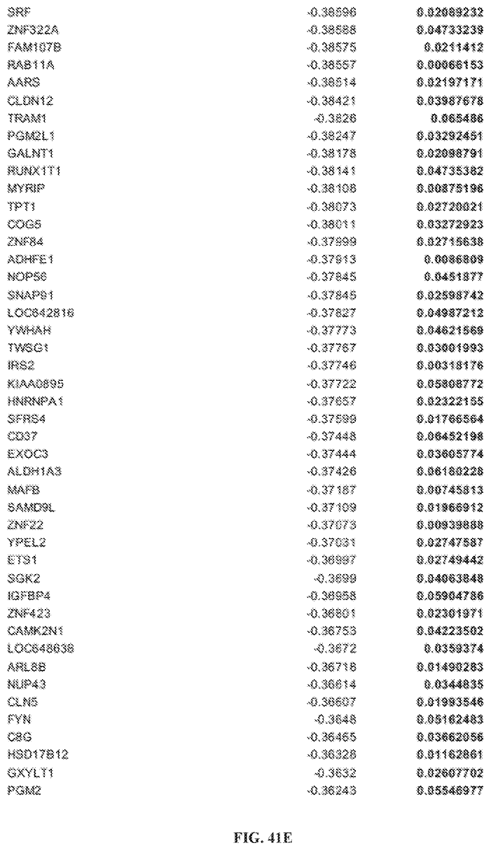

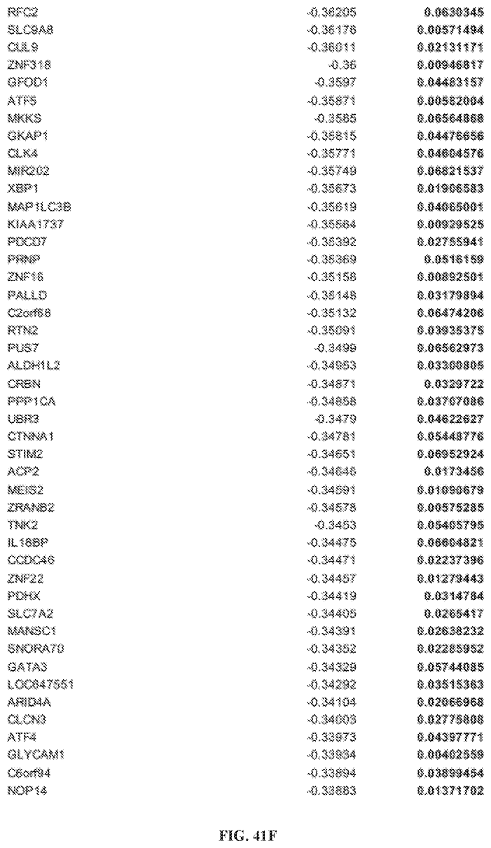

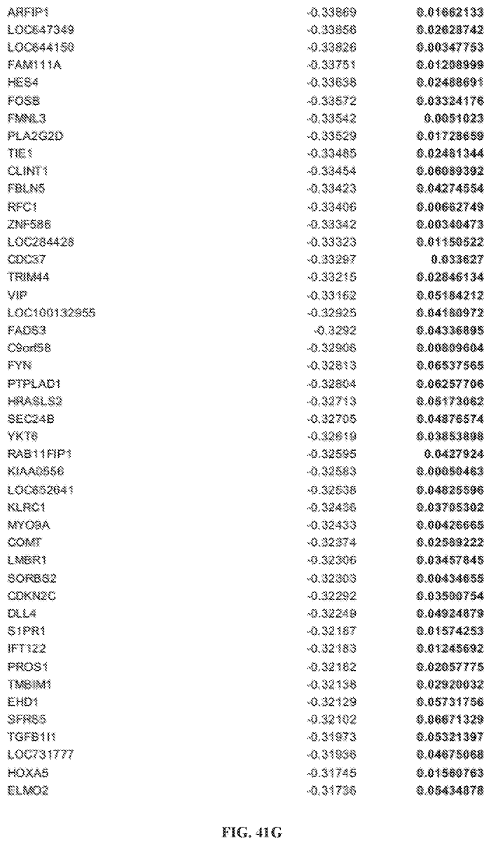

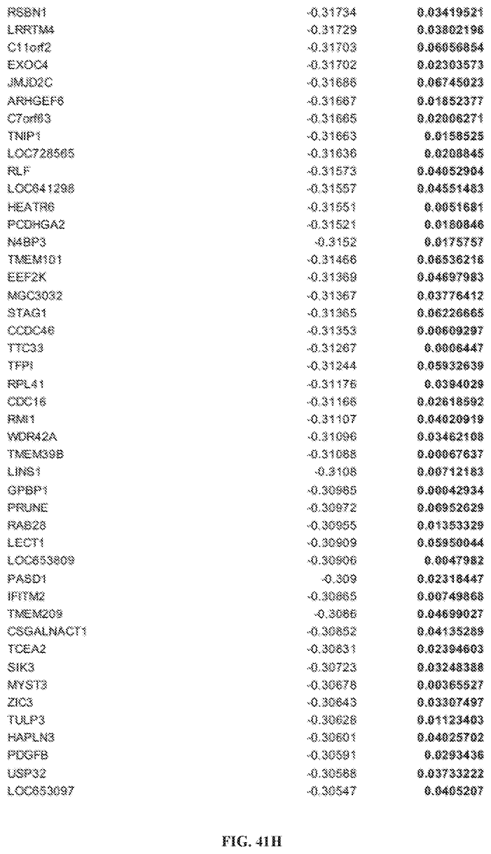

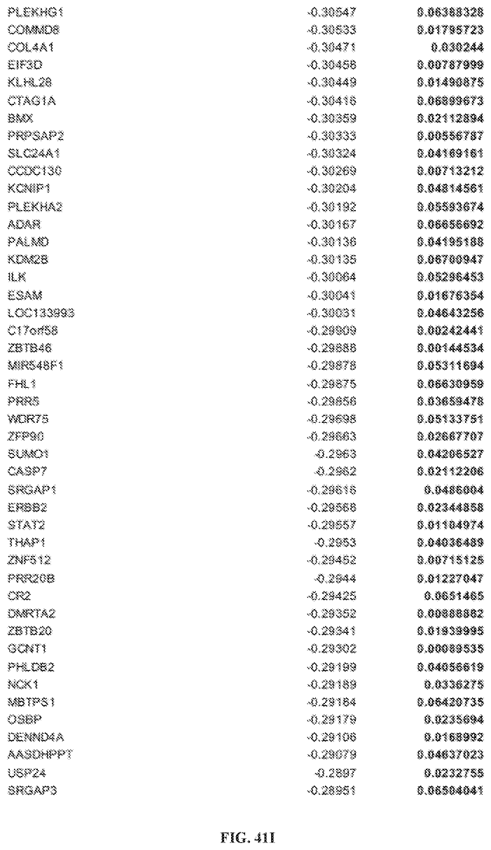

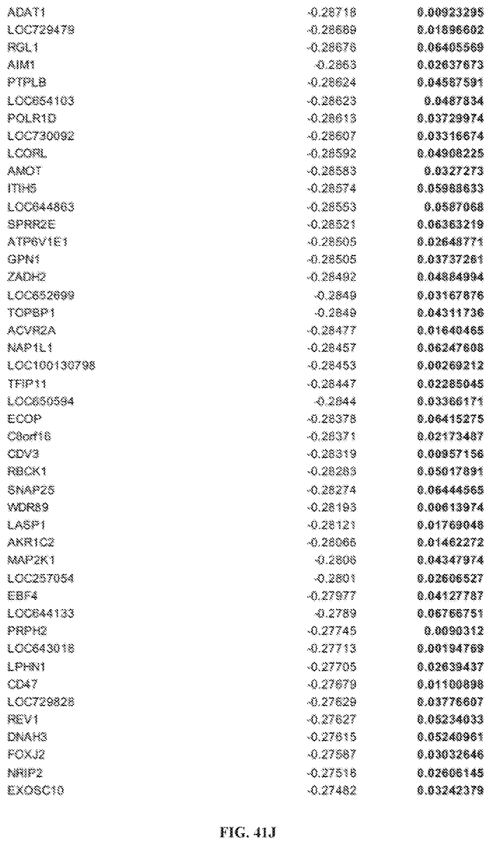

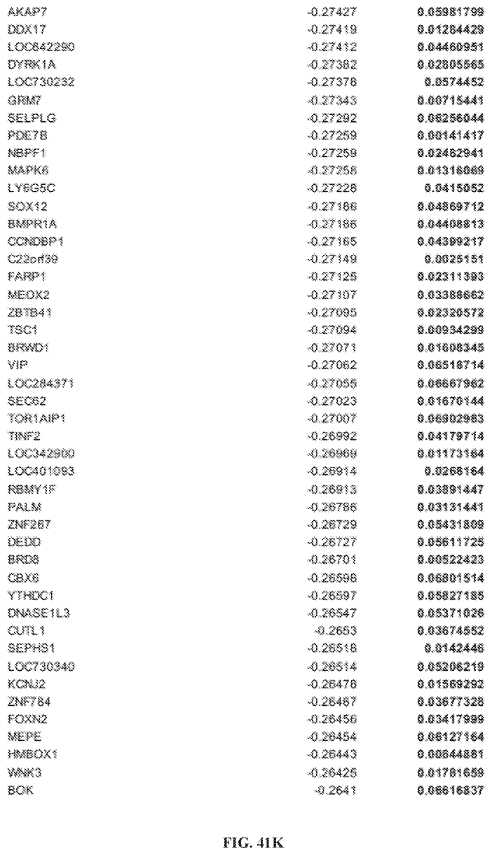

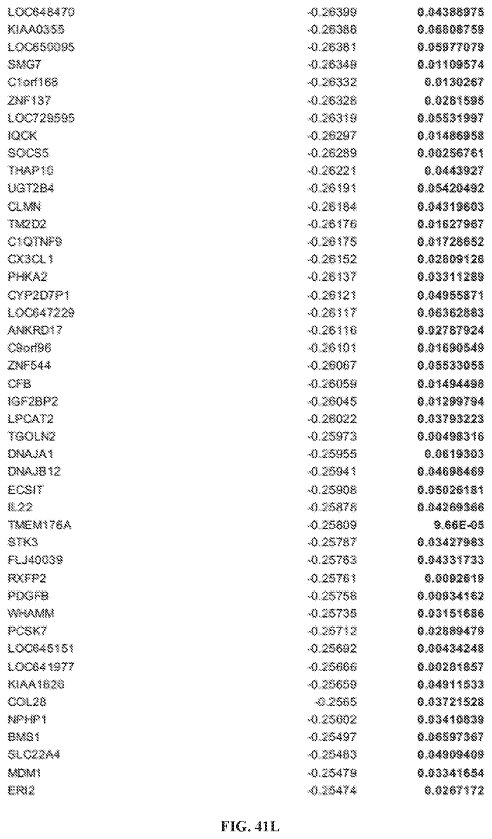

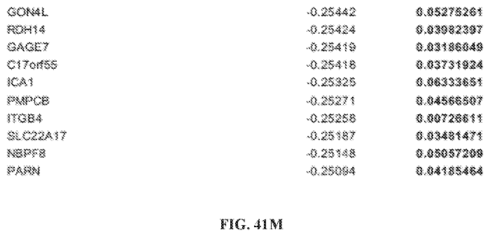

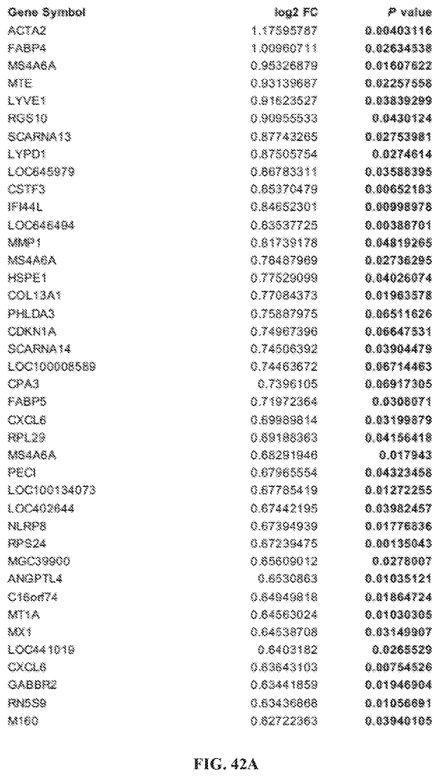

To further investigate the mechanistic links between miR-335 and NF-κB activation, the genes that were significantly dysregulated in our gene expression profile analyses of miR-335 overexpressing HUVECs were cross-referenced to those genes that are predicted to be targeted by miR-335. No components of the main NF-κB transcriptional complex itself were differentially regulated by miR-335 overexpression (including RELA (p65). NFKB1 (p50). REL. NFKB2 (p52), and RELB) (FIG. 33). In silico analyses of an expanded panel of validated NF-κB regulators that were also predicted to be targeted in their 3′ untranslated region (UTR) by miR-335 led to the identification of CHFR (checkpoint with forkhead and ring finger domains), a gene previously described to physically interact with the p65 subdomain of NF-κB complex and downregulate NF-κB target genes. CHFR is a mitotic checkpoint gene that has been found to be silenced in various human malignancies, and Chfr deficient mice are prone to developing cancer, but the function of CHFR in ECs remains largely unknown. CHFR was decreased in response to miR-335 overexpression (FIGS. 41A-41M). It was confirmed by quantitative PCR and western blotting that CHFR is downregulated in response to miR-335 overexpression in HCMECs and HUVECs (FIG. 26A), while its expression was significantly upregulated in response to stimulation with atorvastatin (FIG. 26B). Furthermore, it was confirmed that binding of miR-335 to 3′ UTR of CHFR, which was abrogated upon mutagenesis of the predicted miR-335 binding site (FIG. 26C, 34).

### Example 5

### Determined the Effect of CHFR Modulation on EC Behavior

Similar to the effect of miR-335 overexpression, CHFR silencing in both HCMECs and HUVECs led to significant increase in monocyte adhesion, as well as robust increase in ICAM-1 and E-SELECTIN expression (FIG. 26D, 26 E, 35). Moreover, silencing of CHFR in HUVECs expressing NF-κB-GFP reporter led to a significant increase in GFP expression, further confirming the role of CHFR NF-κB activation (FIG. 26F).

Further, to demonstrate that the miR-335 effect on increased expression of inflammatory markers was dependent on CHFR, miR-335 and CHFR in HUVECs was concurrently overexpressed, and found that the increased expression of ICAM-1 and E-SELECTIN in response to miR-335 overexpression was abrogated by concurrent CHFR expression (FIG. 26G).

### Example 6

### Investigate the Function of miR-335 In Vivo

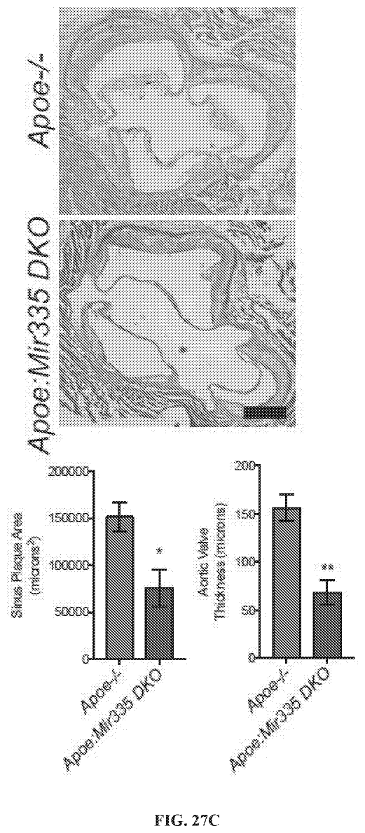

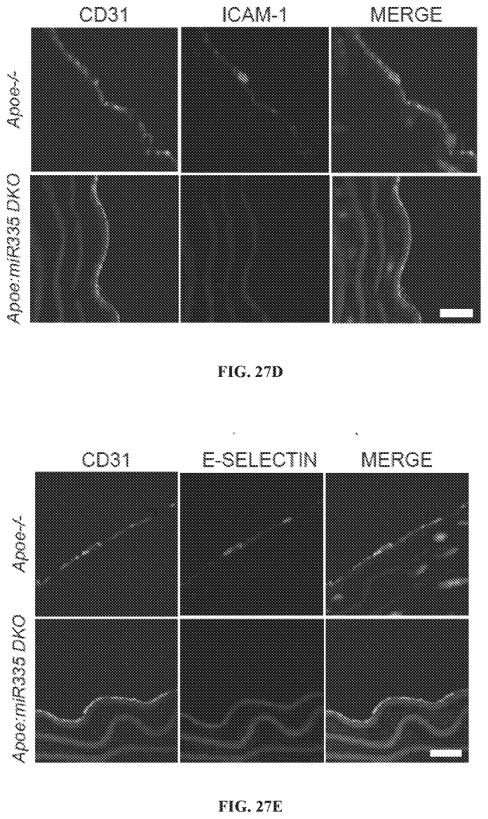

To investigate the function of miR-335 in vivo, expression profiling of miR-335 was first conducted by in situ hybridization, demonstrating expression of miR-335 in ECs in multiple vascular beds, including the aorta, skeletal muscle, and adipose tissue, although basal expression levels appeared to be lower than that of miR-126, a validated endothelial miRNA (FIG. 27A). To further interrogate the function of miR-335 in the vascular disease context, miR-335 null mice was generated on the atherosclerosis susceptible Apoe−/− background (henceforth designated Apoe:Mir335 DKO) (FIG. 36). Mice were fed a high-fat diet for 16 weeks, after which the aortic atherosclerosis burden was quantified. It was found that Apoe:Mir335 DKO mice had significantly less atherosclerotic burden compared to Apoe−/− littermates (FIGS. 27B, 27C). Moreover, it was found that the expression of inflammatory markers ICAM-1 and E-SELECTIN, which were highly expressed in the endothelial layer of Apoe−/− mice on a high-fat diet, was markedly decreased in the Apoe:Mir335 DKO mice (FIG. 27D, 27E).

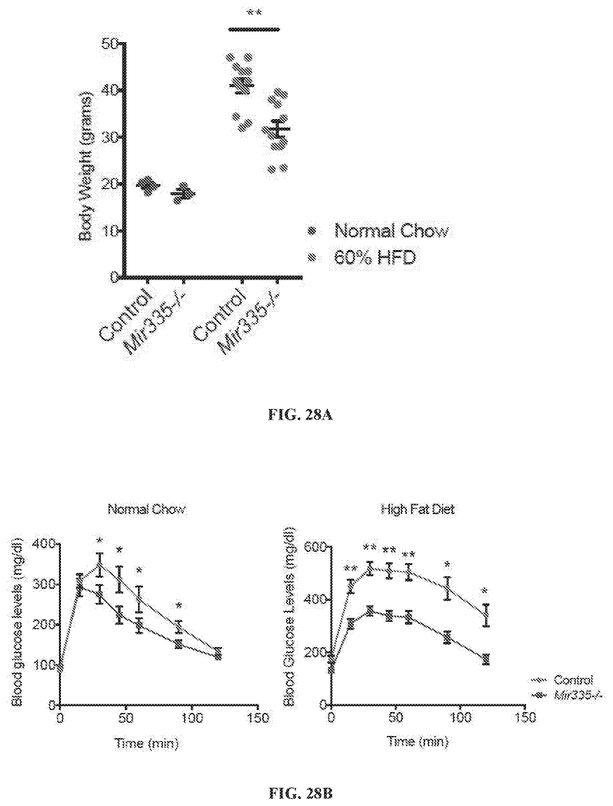

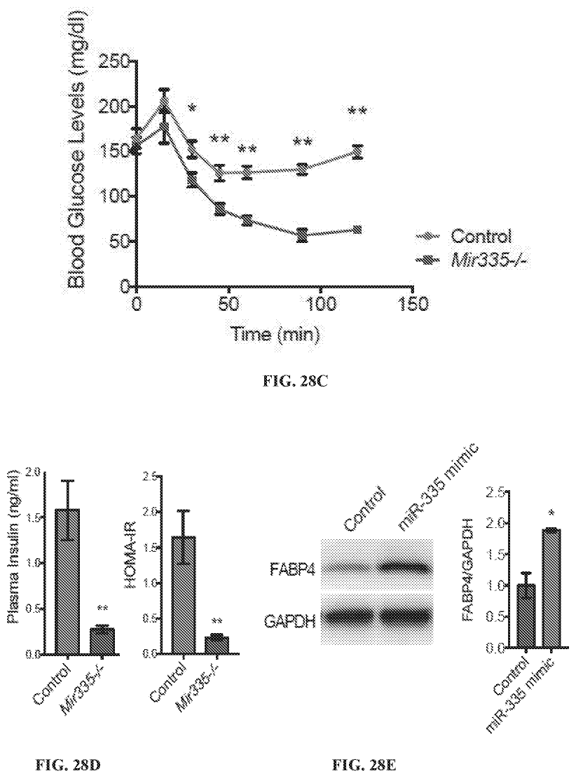

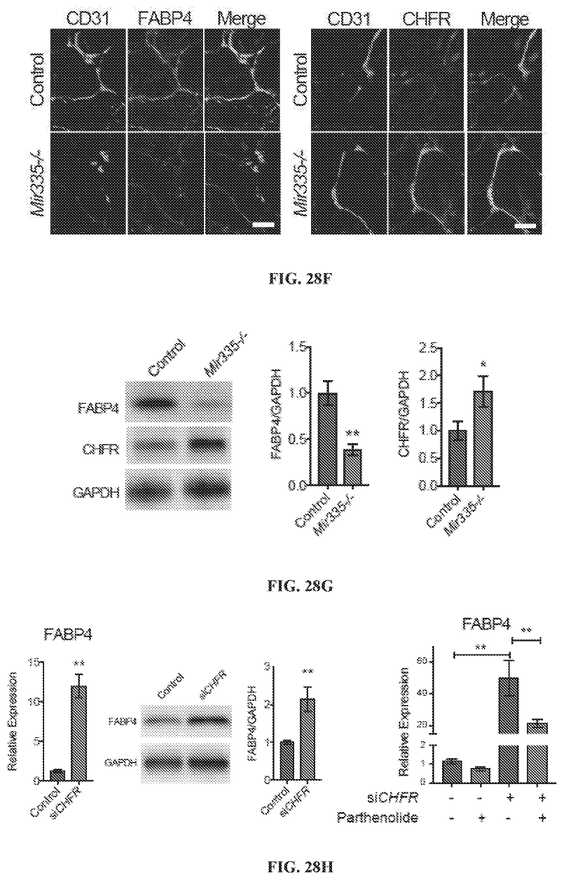

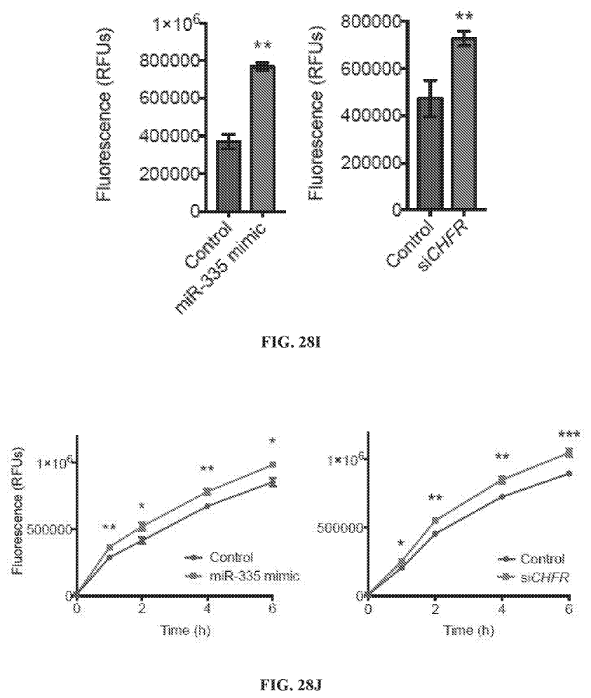

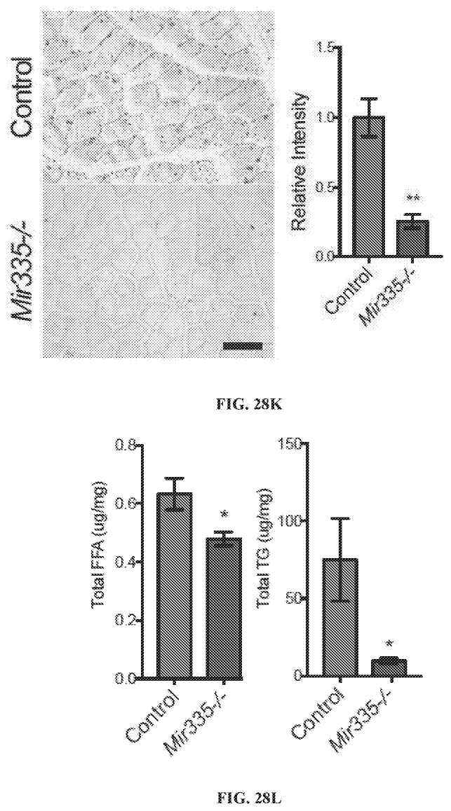

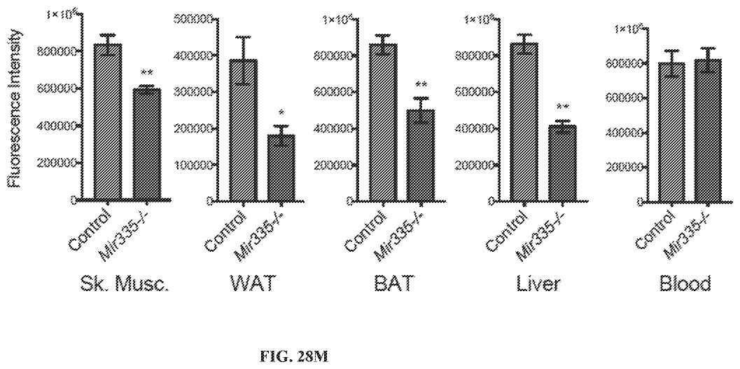

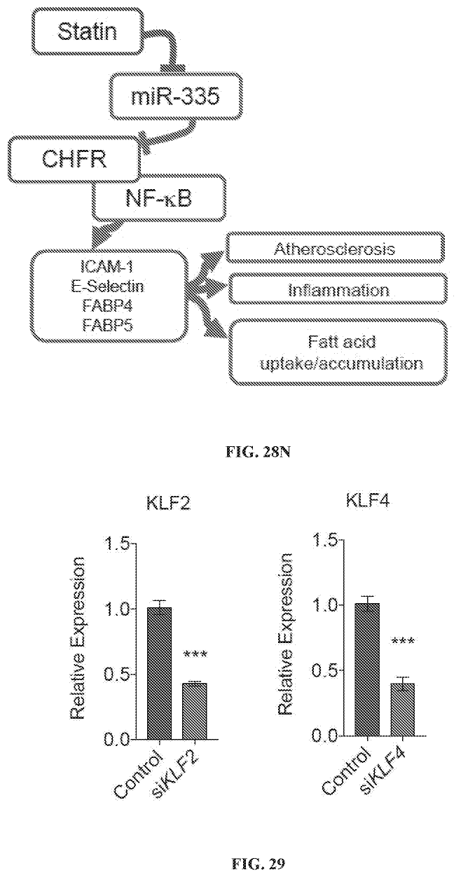

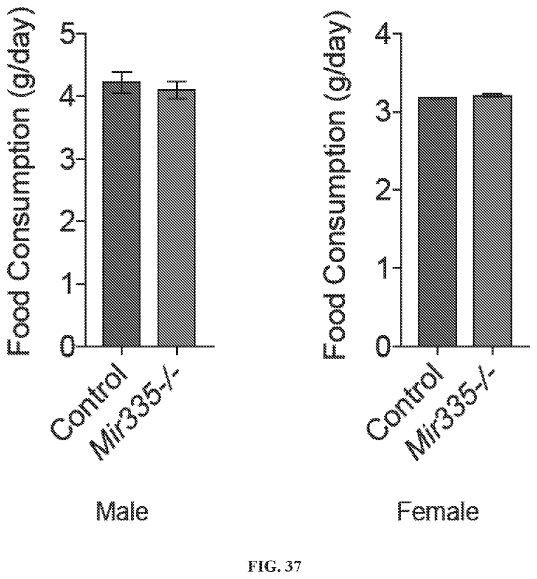

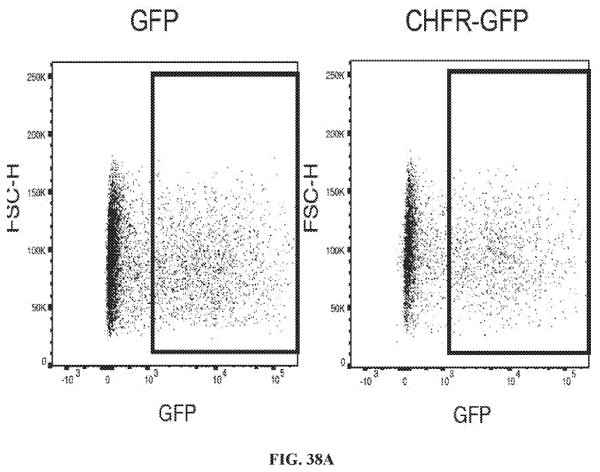

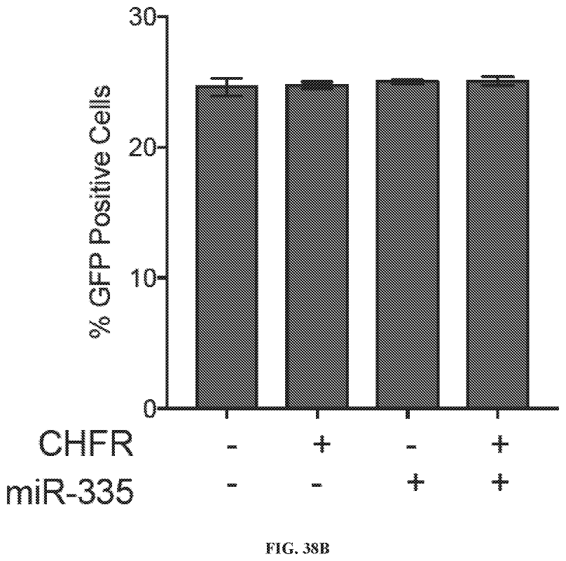

Although Mir335−/− mice were previously described to not have any overt phenotype at baseline, given that lipid metabolism was the most significantly regulated pathway by miR-335, we pursued additional metabolic phenotyping of Mir335−/− mice. The baseline weights of Mir335−/− mice were not significantly different from their control littermates on a normal chow diet. However, it was found that Mir335−/− mice fed a high-fat diet gained significantly less weight compared to their control littermates (FIG. 28A). The intraperitoneal glucose tolerance testing of Mir335−/− mice was conducted, and it was found that these mice have significantly increased glucose utilization compared to controls, and the difference was even more pronounced when these mice were challenged with a high-fat diet (FIG. 28B). Moreover, insulin tolerance testing demonstrated Mir335−/− mice to be significantly more insulin sensitive (FIG. 28C), with significantly lower fasting plasma insulin level and homeostatic model assessment of insulin resistance (HOMA-IR) in the Mir335−/− mice compared to control littermates (FIG. 28D). It was confirmed that the dietary food intake in the Mir335−/− mice was comparable to their control littermates on both normal chow and on a high-fat diet (FIG. 37), excluding change in food intake as the primary cause of the metabolic difference between the mouse strains.

It was next sought to further investigate the mechanism of miR-335's effect on lipid handling and metabolism. It was previously found that increased expression of endothelial FABP4 was a key regulatory mechanism by which trans-endothelial transfer of fatty acid, and overall glucose utilization and insulin sensitivity, is regulated. Given that FABP4 was identified as a gene that is significantly upregulated in ECs with miR-335 overexpression (FIGS. 42A-42K), it was further investigated this as a potential mechanism by which abrogation of miR-335 affords metabolic protection against high-fat diet induced obesity and insulin resistance.

First, it was confirmed that FABP4 expression was significantly increased in ECs subjected to miR-335 overexpression (FIG. 28E). Moreover, it was found that the expression of FABP4 decreased in the skeletal muscle endothelium of Mir335−/− mice (FIGS. 28F, 28G). The reduced FABP4 expression strongly correlated with increased CHFR expression in the endothelium in vivo (FIGS. 28F, 28G). It was found that knockdown of CHFR in HUVECs led to a significant increase in FABP4 expression, whereas treatment of HUVECs with parthenolide, an NF-κB inhibitor, led to inhibition of CHFR knockdown induced increase in FABP4 expression, further supporting the role of NF-κB as an intermediary that regulates FABP4 downstream of miR-335 (FIG. 28H).

It was further sought to investigate the role of fatty acid transfer in Mir335−/− metabolic phenotype. As increased endothelial expression of FABP4 was previously found to augment endothelial fatty acid uptake, as well as trans-endothelial fatty acid transfer, the impact of endothelial miR-335 on expression on fatty acid handling was determined.

Overexpression of miR-335 or knockdown of CHFR in HUVECs led to significantly increased cellular uptake of BODIPY 558/568 C12 (BODIPY) fatty acid in HUVECs (FIG. 28I).

Moreover, trans-endothelial BODIPY transfer was also significantly increased when Transwell assay was conducted using HUVECs with either overexpression of miR-335 or knockdown of CHFR (FIG. 28J). In vivo, it was found that Oil Red O staining of skeletal muscle was significantly higher in control mice compared to Mir335−/− mice (FIG. 28K). Fatty acid and triglyceride content of the skeletal muscle was also significantly decreased in Mir335−/− mice (FIG. 28L), suggesting that inhibition of fatty acid accumulation in the skeletal muscle may be a key mechanism impacting overall body glucose utilization and insulin sensitivity in Mir335−/− mice. Lastly, oral gavage administration of fluorescently labeled fatty acid led to significantly decreased accumulation in multiple metabolic tissues of Mir335−/− mice, including the skeletal muscle, adipose tissues, and the liver, while circulating serum fluorescent fatty acid was comparable between the two strains, suggesting impaired 5 trans-endothelial fatty acid transfer, but preserved gut absorption in Mir335−/− mice (FIG. 28M). These findings further implicate trans-endothelial fatty acid transport as a key process that is directly targeted by miR-335.

### Example 7

The studies conducted herein identify a previously undefined downstream target of statins, namely downregulation of miR-335, as a key mechanism of both vascular inflammation and metabolic homeostasis (FIG. 28N). While expression of miR-335 has been found to be increased in metabolically challenged states such as obesity, whether it has disease-promoting roles in such contexts has not been described to date. The demonstration that genetic deletion of miR-335 in mice leads to decreased atherosclerosis and improvements in multiple metabolic parameters, including decreased body weight, improved glucose utilization, and improved insulin sensitivity, reveals important disease-relevant roles for this miRNA. Further studies to therapeutically downregulate or inhibit its activity may provide a novel strategy to improve our management of vascular and metabolic disease states

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or ameliorating a disease or disorder selected from the group consisting of non-alcoholic steatohepatitis (NASH), non-alcoholic fatty liver disease (NAFLD), diabetes, atherosclerosis, and obesity in a subject, the method comprising administering to the subject a therapeutically effective amount of at least one miRNA335 inhibitor.

2-5. (canceled)

6. The method according to claim 1, wherein the at least one miRNA335 inhibitor comprises an antagomir.

7. The method according to claim 1, wherein the subject is a mammal.

8. The method according to claim 1, wherein the subject is a human.

9. The method according to claim 1, wherein the at least one miRNA335 inhibitor is formulated as a pharmaceutical composition further comprising at least one pharmaceutically acceptable carrier.


SEQUENCE LISTING (5 sequences)

SEQ ID NO: 1 - DNA, 94 bp, Homo sapiens
tgttttgagcgggggtcaagagcaataacgaaaaatgtttgtcataaaccgtttttcattattgctcctgacctcctctcatttgctatattca

SEQ ID NO: 2 - DNA, 11 bp, Artificial Sequence
  NF-kB response elements
gggaatttccc

SEQ ID NO: 3 - DNA, 23 bp, Artificial Sequence
  miR-335-5p
acatttttcgttattgctcttga

SEQ ID NO: 4 - DNA, 20 bp, Artificial Sequence
  miR-126-3p
cattattactcacggtacga

SEQ ID NO: 5 - DNA, 21 bp, Artificial Sequence
  Scrambled probe
tgtaacacgtctatacgccca